SEQUENCE LISTING (74 sequences)

SEQ ID NO: 1 - DNA, 30 bp, Artificial Sequence
  CDS 1..30: completely synthesized
tttgccaaaaaatttgccaaaaaatttaaa

SEQ ID NO: 2 - DNA, 60 bp, Homo sapiens
  CDS 1..60: Endostatin Fragment
attgttcgtcgtgctgatcgtgctgctgttcctattgttaatttgaaagatgaattgttg

SEQ ID NO: 3 - DNA, 60 bp, Homo sapiens
  CDS 1..60: PL-2
atgttcggaaacggaaagggatacagaggaaagagagcaacaacagtaacaggaacacca

SEQ ID NO: 4 - DNA, 57 bp, Artificial Sequence
  completely synthesized
ttcgcaaagaagttcgcaaagaagttcaagaagttcgcaaagttcgcattcgcattc

SEQ ID NO: 5 - DNA, 69 bp, Artificial Sequence
  CDS 1..69: completely synthesized
ttcgcgaagaagttcgcgaagaagttcaagaagttcgcgaagaagttcgcgaagttcgcgttcgcgttc

SEQ ID NO: 6 - DNA, 45 bp, Artificial Sequence
  CDS 1..45: completely synthesized
ttcgcaaagaagttcgcaaagaagttcgcaaagaagttcgcaaag

SEQ ID NO: 7 - DNA, 45 bp, Artificial Sequence
  CDS 1..45: completely synthesized
aagaagttcaagaagttcgcaaagaagttcgcaaagttcgcattc

SEQ ID NO: 8 - DNA, 36 bp, Artificial Sequence
  CDS 1..36: completely synthesized
tttgctaaaaaatttgctaaaaaatttaaaaaattt

SEQ ID NO: 9 - DNA, 78 bp, Apis melifera
  CDS 1..78: Melittin
gggataggggcggtgctgaaggtgctgacgacggggctgccggcgctgataagctggataaagaggaagaggcagcag

SEQ ID NO: 10 - DNA, 75 bp, Rana pipens
  CDS 1..75: Pipinin
ttcctaccgataatagcgggggtggcggcgaaggtgctattcccgaagatattctgcgcgataagtaagaagtgc

SEQ ID NO: 11 - DNA, 99 bp, Homo sapiens
  CDS 1..99: Adenoregulin
ggactctggtcgaagatcaaggaggtaggaaaggaggcagcaaaggcagcagcaaaggcagcaggaaaggcagcactcggagcagtatcggaggcagta

SEQ ID NO: 12 - DNA, 105 bp, Hyalophora cecropia
  CDS 1..105: Cecropin B
aaatggaaaatttttaaaaaaattgaaaaagttggtcgtaatattcgtaatggtattattaaagctggtcctgctgttgctgttttgggtgaagctaaagctttg

SEQ ID NO: 13 - DNA, 102 bp, Homo sapiens
  CDS 1..102: Andropin
gtttttattgatattttggataaagttgaaaatgctattcataatgctgctcaagttggtattggttttgctaaaccttttgaaaaattgattaatcctaaa

SEQ ID NO: 14 - DNA, 69 bp, Xenopus laevis
  CDS 1..69: Magainin II
ggcatcggcaaatttcttcattcagccaaaaaatttggcaaagcctttgtcggcgaaatcatgaattca

SEQ ID NO: 15 - DNA, 69 bp, Artificial Sequence
  CDS 1..69: completely synthesized
ttcgcgttcgcgttcaaggcgttcaagaaggcgttcaagaagttcaagaaggcgttcaagaaggcgttc

SEQ ID NO: 16 - DNA, 69 bp, Artificial Sequence
  CDS 1..69: completely synthesized
ttcgtgaagaaggtggcgaagaaggcgaagaaggtggcgaagaaggcggtgaaggtggcgaagaaggtg

SEQ ID NO: 17 - DNA, 114 bp, Homo sapiens
  CDS 1..114: beta defensin 1
gattttgcctcatgtcataccaatggcggcatctgtcttcccaatcgatgtcccggccatatgatccaaatcggcatctgttttcgaccccgagtcaaatgttgtcgatcatgg

SEQ ID NO: 18 - DNA, 51 bp, Artificial Sequence
  CDS 1..51: completely synthesized
ttcaagctgagggcgaagataaaggtgaggctgagggcgaagataaagctg

SEQ ID NO: 19 - DNA, 54 bp, Homo sapiens
  CDS 1..54: Protegrin
cggggaggacggctctgctactgccggcggcggttctgcgtatgcgtaggacgg

SEQ ID NO: 20 - DNA, 2430 bp, Homo sapiens
  CDS 1..2430: Plasminogen
atggagcacaaggaggtagtactcctcctcctcctcttcctcaagtcgggacagggagagccactcgacgactacgtaaacacacagggagcatcgctcttctcggtaacaaagaagcagctcggagcaggatcgatcgaggagtgcgcagcaaagtgcgaggaggacgaggagttcacatgccgggcattccagtaccactcgaaggagcagcagtgcgtaatcatggcagagaaccggaagtcgtcgatcatcatccggatgcgggacgtagtactcttcgagaagaaggtatacctctcggagtgcaagacaggaaacggaaagaactaccggggaacaatgtcgaagacaaagaacggaatcacatgccagaagtggtcgtcgacatcgccacaccggccacggttctcgccagcaacacacccatcggagggactcgaggagaactactgccggaacccagacaacgacccacagggaccatggtgctacacaacagacccagagaagcggtacgactactgcgacatcctcgagtgcgaggaggagtgcatgcactgctcgggagagaactacgacggaaagatctcgaagacaatgtcgggactcgagtgccaggcatgggactcgcagtcgccacacgcacacggatacatcccatcgaagttcccaaacaagaacctcaagaagaactactgccggaacccagaccgggagctccggccatggtgcttcacaacagacccaaacaagcggtgggagctctgcgacatcccacggtgcacaacaccaccaccatcgtcgggaccaacataccagtgcctcaagggaacaggagagaactaccggggaaacgtagcagtaacagtatcgggacacacatgccagcactggtcggcacagacaccacacacacacaaccggacaccagagaacttcccatgcaagaacctcgacgagaactactgccggaacccagacggaaagcgggcaccatggtgccacacaacaaactcgcaggtacggtgggagtactgcaagatcccatcgtgcgactcgtcgccagtatcgacagagcagctcgcaccaacagcaccaccagagctcacaccagtagtacaggactgctaccacggagacggacagtcgtaccggggaacatcgtcgacaacaacaacaggaaagaagtgccagtcgtggtcgtcgatgacaccacaccggcaccagaagacaccagagaactacccaaacgcaggactcacaatgaactactgccggaacccagacgcagacaagggaccatggtgcttcacaacagacccatcggtacggtgggagtactgcaacctcaagaagtgctcgggaacagaggcatcggtagtagcaccaccaccagtagtactcctcccagacgtagagacaccatcggaggaggactgcatgttcggaaacggaaagggataccggggaaagcgggcaacaacagtaacaggaacaccatgccaggactgggcagcacaggagccacaccggcactcgatcttcacaccagagacaaacccacgggcaggactcgagaagaactactgccggaacccagacggagacgtaggaggaccatggtgctacacaacaaacccacggaagctctacgactactgcgacgtaccacagtgcgcagcaccatcgttcgactgcggaaagccacaggtagagccaaagaagtgcccaggacgggtagtaggaggatgcgtagcacacccacactcgtggccatggcaggtatcgctccggacacggttcggaatgcacttctgcggaggaacactcatctcgccagagtgggtactcacagcagcacactgcctcgagaagtcgccacggccatcgtcgtacaaggtaatcctcggagcacaccaggaggtaaacctcgagccacacgtacaggagatcgaggtatcgcggctcttcctcgagccaacacggaaggacatcgcactcctcaagctctcgtcgccagcagtaatcacagacaaggtaatcccagcatgcctcccatcgccaaactacgtagtagcagaccggacagagtgcttcatcacaggatggggagagacacagggaacattcggagcaggactcctcaaggaggcacagctcccagtaatcgagaacaaggtatgcaaccggtacgagttcctcaacggacgggtacagtcgacagagctctgcgcaggacacctcgcaggaggaacagactcgtgccagggagactcgggaggaccactcgtatgcttcgagaaggacaagtacatcctccagggagtaacatcgtggggactcggatgcgcacggccaaacaagccaggagtatacgtacgggtatcgcggttcgtaacatggatcgagggagtaatgcggaacaac

SEQ ID NO: 21 - DNA, 768 bp, Homo sapiens
  CDS 1..768: Angiostatin
aaggtgtacctgagcgagtgcaagacggggaacgggaagaactacagggggacgatgagcaagacgaagaacgggataacgtgccagaagtggagcagcacgagcccgcacaggccgaggttcagcccggcgacgcacccgagcgaggggctggaggagaactactgcaggaacccggacaacgacccgcaggggccgtggtgctacacgacggacccggagaagaggtacgactactgcgacatactggagtgcgaggaggagtgcatgcactgcagcggggagaactacgacgggaagataagcaagacgatgagcgggctggagtgccaggcgtgggacagccagagcccgcacgcgcacgggtacataccgagcaagttcccgaacaagaacctgaagaagaactactgcaggaacccggacagggagctgaggccgtggtgcttcacgacggacccgaacaagaggtgggagctgtgcgacataccgaggtgcacgacgccgccgccgagcagcgggccgacgtaccagtgcctgaaggggacgggggagaactacagggggaacgtggcggtgacggtgagcgggcacacgtgccagcactggagcgcgcagacgccgcacacgcacaacaggacgccggagaacttcccgtgcaagaacctggacgagaactactgcaggaacccggacgggaagagggcgccgtggtgccacacgacgaacagccaggtgaggtgggagtactgcaagataccgagctgc

SEQ ID NO: 22 - DNA, 81 bp, Homo sapiens
  CDS 1..81: PL-1
caagcttgggattctcaatctcctcatgctcatggttatattccttctaaatttcctaataaaaatttgaaaaaaaattat

SEQ ID NO: 23 - DNA, 2052 bp, Homo sapiens
  CDS 1..2052: Collagen Fragment
ggagaggtaggagcagacggaatcccaggattcccaggactcccaggacgggagggaatcgcaggaccacagggaccaaagggagaccggggatcgcggggagagaagggagacccaggaaaggacggactcggacagccaggactcccaggaccacggggaccaccaggaccagtagtatacgtatcggagcaggacggatcggtactctcggtaccaggaccagagggacggcggggattcgcaggattcccaggaccagcaggaccaaagggaaacctcggatcgaagggagagctcggatcgccaggaccaaagggagagaagggagagccaggatcgatcttctcgccagacggaggagcactcggaccagcacagaagggagcaaagggagagccaggattccggggaccaccaggactctacggacggccaggatacaagggagagatcggattcccaggacggccaggacggccaggaatgaacggactcaagggagagaagggagagccaggagacgcatcgctcggattcggaatgcggggaatgccaggaccaccaggaccaccaggaccaccaggaccaccaggaacaccagtatacgactcgaacgtattcgcagagtcgtcgcggccaggaccaccaggactcccaggaaaccagggaccaccaggaccaaagggaccaaagggagaggtaggaccaccaggaccaccaggacagttcccattcgacttcctccagaaggaggcagagatgaagggagagaagggagaccggggagacgcaggacagaagggagagcggggagagccaggaggaggaggattcttcggatcgtcgctcccaggagcaccaggagcaccaggaccacggggatacccaggaatcccaggaccaaagggagagtcgatccggggacagccaggaccaccaggaccacagggaccaccaggaatcggatacgagggacggcagggaccaccaggaccaccaggaccaccaggaccaccatcgttcccaggaccacaccggcagacaatctcggtaccaggaccaccaggaccaccaggaccaccaggaccaccaggaacaatgggagcatcgtcgggacaggtacggctctgggcaacacggcaggcaatgctcggacaggtacacgaggtaccagagggatggctcatcttcgtagcagagcaggaggagctctacgtacgggtacagaacggattccggaaggtacagctcgaggcacggacaccactcccacggggaacagacaacgaggtagcagcactccagccaccagtagtacagctccacgactcgaacccatacccacggcgggagcacccacacccaacagcacggccatggcgggcagacgacatcctcgcatcgccaccaggactcccagagccacagccatacccaggaggaccacaccactcgtcgtacgtacactgcggaccagcacggccaacatcgccaccagcacactcgcaccgggacttccagccagtactccacctcgtagcactcaactcgccactctcgggaggaatgcggggaatccggggagcagacttccagtgcttccagcaggcacgggcagtaggactcgcaggaacattccgggcattcctctcgtcgcggctccaggacctctactcgatcgtacggcgggcagaccgggcagcagtaccaatcgtaaacctcaaggacgagctcctcttcccatcgtgggaggcactcttctcgggatcggagggaccactcaagccaggagcacggatcttctcgttcgacggaaaggacgtactccggcacccaacatggccacagaagtcggtatggcacggatcggacccaaacggacggcggctcacagagtcgtactgcgagacatggcggacagaggcaccatcggcaacaggacaggcatcgtcgctcctcggaggacggctcctcggacagtcggcagcatcgtgccaccacgcatacatcgtactctgcatcgagaactcgttcatgacagcatcgaag

SEQ ID NO: 24 - DNA, 63 bp, Homo sapiens
  CDS 1..63: C-1
atcgtacggcgggcagaccgggcagcagtaccaatcgtaaacctcaaggacgagctcctcttc

SEQ ID NO: 25 - DNA, 210 bp, Homo sapiens
  CDS 1..210: Platelet Factor 4
gaggcggaggaggacggggacctgcagtgcctgtgcgtgaagacgacgagccaggtgaggccgaggcacataacgagcctggaggtgataaaggcggggccgcactgcccgacggcgcagctgatagcgacgctgaagaacgggaggaagatatgcctggacctgcaggcgccgctgtacaagaagataataaagaagctgctggagagc

SEQ ID NO: 26 - DNA, 45 bp, Homo sapiens
  CDS 1..45: Platelet Factor 4 Fragment 1
cccaccgcccaattaattgccaccttaaaaaatggccgcaaaatt

SEQ ID NO: 27 - DNA, 54 bp, Homo sapiens
  CDS 1..54: Platelet Factor 4 Fragment 2
ttggatttgcaagctcctttgtataaaaaaattattaaaaaattgttggaatct

SEQ ID NO: 28 - DNA, 54 bp, Artificial Sequence
  CDS 1..54: completely synthesized
ttcgcgaagaagttcgcgaagaagttcaagaagttcgcgaagaagttcgcgaag

SEQ ID NO: 29 - DNA, 45 bp, Artificial Sequence
  CDS 1..45: completely synthesized
aaaaaatttaaaaaatttgccaaaaaatttgccaaatttgccttt

SEQ ID NO: 30 - DNA, 30 bp, Artificial Sequence
  CDS 1..30: completely synthesized
ttcgcgaagaagttcgcgaagttcgcgttc

SEQ ID NO: 31 - DNA, 297 bp, Homo sapiens
  CDS 1..297: IL8
atgacaagtaagctagcagtagcactactagcagcattcctaataagtgcagcactatgcgagggagcagtactaccaagaagtgcaaaggagctaagatgccagtgcataaagacatacagtaagccattccacccaaagttcataaaggagctaagagtaatagagagtggaccacactgcgcaaacacagagataatagtaaagctaagtgacggaagagagctatgcctagacccaaaggagaactgggtacagagagtagtagagaagttcctaaagagagcagagaacagt

SEQ ID NO: 32 - DNA, 375 bp, Homo sapiens
  CDS 1..375: MIG
atgaaaaaatctggtgttttgtttttgttgggtattattttgttggttttgattggtgttcaaggtactcctgttgttcgtaaaggtcgttgttcttgtatttctactaatcaaggtactattcatttgcaatctttgaaagatttgaaacaatttgctccttctccttcttgtgaaaaaattgaaattattgctactttgaaaaatggtgttcaaacttgtttgaatcctgattctgctgatgttaaagaattgattaaaaaatgggaaaaacaagtttctcaaaaaaaaaaacaaaaaaatggtaaaaaacatcaaaaaaaaaaagttttgaaagttcgtaaatctcaacgttctcgtcaaaaaaaaactact

SEQ ID NO: 33 - DNA, 294 bp, Homo sapiens
  CDS 1..294: IP-10
atgaatcaaactgctattttgatttgttgtttgatttttttgactttgtctggtattcaaggtgttcctttgtctcgtactgttcgttgtacttgtatttctatttctaatcaacctgttaatcctcgttctttggaaaaattggaaattattcctgcttctcaattttgtcctcgtgttgaaattattgctactatgaaaaaaaaaggtgaaaaacgttgtttgaatcctgaatctaaagctattaaaaatttgttgaaagctgtttctaaagaacgttctaaacgttctcct

SEQ ID NO: 34 - DNA, 297 bp, Homo sapiens
  CDS 1..297: MCP-1
atgaaggtatcggcagcactcctctgcctcctcctcatcgcagcaacattcatcccacagggactcgcacagccagacgcaatcaacgcaccagtaacatgctgctacaacttcacaaaccggaagatctcggtacagcggctcgcatcgtaccggcggatcacatcgtcgaagtgcccaaaggaggcagtaatcttcaagacaatcgtagcaaaggagatctgcgcagacccaaagcagaagtgggtacaggactcgatggaccacctcgacaagcagacacagacaccaaagaca

SEQ ID NO: 35 - DNA, 276 bp, Homo sapiens
  CDS 1..276: MIP-1 alpha 2
atgcaggtatcgacagcagcactcgcagtactcctctgcacaatggcactctgcaaccagttctcggcatcgctcgcagcagacacaccaacagcatgctgcttctcgtacacatcgcggcagatcccacagaacttcatcgcagactacttcgagacatcgtcgcagtgctcgaagccaggagtaatcttcctcacaaagcggtcgcggcaggtatgcgcagacccatcggaggagtgggtacagaagtacgtatcggacctcgagctctcggca

SEQ ID NO: 36 - DNA, 273 bp, Homo sapiens
  CDS 1..273: RANTES
atgaaagtctcagccgccgcccttgccgtcatccttatcgccaccgccctttgtgcccccgcctcagcctcaccctattcatcagataccaccccctgttgttttgcctatatcgcccgaccccttccccgagcccatatcaaagaatatttttatacctcaggcaaatgttcaaatcccgccgtcgtctttgtcacccgaaaaaatcgacaagtctgtgccaatcccgaaaaaaaatgggtccgagaatatatcaattcacttgaaatgtca

SEQ ID NO: 37 - DNA, 30 bp, Homo sapiens
  CDS 1..30: RANTES Fragment
tgggtgagggagtacataaacagcctggag

SEQ ID NO: 38 - DNA, 27 bp, Homo sapiens
  CDS 1..27: CCL8 Fragment
tgggtacgggactcgatgaagcacctc

SEQ ID NO: 39 - DNA, 27 bp, Homo sapiens
  CDS 1..27: CCL11 Fragment
aaaaaatgggttcaagattccatgaaa

SEQ ID NO: 40 - DNA, 27 bp, Homo sapiens
  CDS 1..27: CC12 Fragment
tgggtcaaaaattcaatcaatcatctt

SEQ ID NO: 41 - DNA, 27 bp, Homo sapiens
  CDS 1..27: CCL13 Fragment
tgggtgcagaactacatgaagcacctg

SEQ ID NO: 42 - DNA, 30 bp, Homo sapiens
  CDS 1..30: CCL14 Fragment
aagtgggtgcaggactacataaaggacatg

SEQ ID NO: 43 - DNA, 30 bp, Homo sapiens
  CDS 1..30: CCL15 Fragment
ttaactaaaaaaggtcgccaagtttgtgct

SEQ ID NO: 44 - DNA, 27 bp, Homo sapiens
  CDS 1..27: CCL16 Fragment
aaacgtgttaaaaatgctgttaaatat

SEQ ID NO: 45 - DNA, 30 bp, Homo sapiens
  CDS 1..30: CCL18 Fragment 1
ttaaccaaacgcggccgccaaatttgtgcc

SEQ ID NO: 46 - DNA, 27 bp, Homo sapiens
  CDS 1..27: CCL18 Fragment 2
aaaaaatgggttcaaaaatatatttct

SEQ ID NO: 47 - DNA, 30 bp, Homo sapiens
  CDS 1..30: CCL19 Fragment
tgggtagagcggatcatccagcggctccag

SEQ ID NO: 48 - DNA, 27 bp, Homo sapiens
  CDS 1..27: CCL23 Fragment
cttaccaaaaaaggccgacgattttgt

SEQ ID NO: 49 - DNA, 30 bp, Homo sapiens
  CDS 1..30: CCL27 Fragment
ctgagcgacaagctgctgaggaaggtgata

SEQ ID NO: 50 - DNA, 30 bp, Homo sapiens
  CDS 1..30: CCL28 Fragment
gtctcccatcatatttcccgccgcttatta

SEQ ID NO: 51 - DNA, 30 bp, Homo sapiens
  CDS 1..30: XCL2 Fragment
tgggtccgcgatgtcgtccgctccatggat

SEQ ID NO: 52 - DNA, 30 bp, Homo sapiens
  CDS 1..30
  CX3CL1 Fragment
tgggtaaaggacgcaatgcagcacctcgac

SEQ ID NO: 53 - DNA, 27 bp, Homo sapiens
  CDS 1..27: CXCL1 Fragment
atggtcaaaaaaatcatcgaaaaaatg

SEQ ID NO: 54 - DNA, 30 bp, Homo sapiens
  CDS 1..30: CXCL3 Fragment
atggtccaaaaaattattgaaaaaatttta

SEQ ID NO: 55 - DNA, 30 bp, Homo sapiens
  CDS 1..30: CXCL4 Fragment
ctgtacaagaagataataaagaagctgctg

SEQ ID NO: 56 - DNA, 30 bp, Homo sapiens
  CDS 1..30: CXCL5 Fragment
tttcttaaaaaagtcatccaaaaaatcctt

SEQ ID NO: 57 - DNA, 30 bp, Homo sapiens
  CDS 1..30: CXCL6 Fragment
ttcctcaagaaggtaatccagaagatcctc

SEQ ID NO: 58 - DNA, 30 bp, Homo sapiens
  CDS 1..30: CXCL7 Fragment
attaaaaaaattgttcaaaaaaaattagct

SEQ ID NO: 59 - DNA, 30 bp, Homo sapiens
  CDS 1..30: CXCL8 Fragment
tgggtccaacgagtcgtcgaaaaatttctt

SEQ ID NO: 60 - DNA, 30 bp, Homo sapiens
  CDS 1..30: CXCL10 Fragment
gcgataaagaacctgctgaaggcggtgagc

SEQ ID NO: 61 - DNA, 21 bp, Homo sapiens
  CDS 1..21: CXCL11 Fragment
atcatcaagaaggtagagcgg

SEQ ID NO: 62 - DNA, 30 bp, Homo sapiens
  CDS 1..30: CXCL13 Fragment
tggatacagagaatgatggaggtgctaaga

SEQ ID NO: 63 - DNA, 30 bp, Homo sapiens
  CDS 1..30: IL10 Fragment
gctgttgaacaagttaaaaatgcttttaat

SEQ ID NO: 64 - DNA, 30 bp, Homo sapiens
  CDS 1..30: IL5 Fragment
acggtggagaggctgttcaagaacctgagc

SEQ ID NO: 65 - DNA, 27 bp, Homo sapiens
  CDS 1..27: IL7 Fragment
tttttgaaacgtttgttgcaagaaatt

SEQ ID NO: 66 - DNA, 24 bp, Homo sapiens
  CDS 1..24: XCL2 Fragment
ttagatcgcttattacgccgctta

SEQ ID NO: 67 - DNA, 24 bp, Homo sapiens
  CDS 1..24: IL20 Fragment
ctcctccggcacctcctccggctc

SEQ ID NO: 68 - DNA, 27 bp, Homo sapiens
  CDS 1..27: IL22 Fragment
aaagatactgttaaaaaattgggtgaa

SEQ ID NO: 69 - DNA, 30 bp, Homo sapiens
  CDS 1..30: IL24 Fragment
ttatttcgccgcgcctttaaacaattagat

SEQ ID NO: 70 - DNA, 30 bp, Homo sapiens
  CDS 1..30: IL26 Fragment
tggattaaaaaattattagaatcctcccaa

SEQ ID NO: 71 - DNA, 24 bp, Homo sapiens
  CDS 1..24: Oncostatin Fragment
tctcgtaaaggtaaacgtttgatg

SEQ ID NO: 72 - DNA, 30 bp, Homo sapiens
  CDS 1..30: Endostatin Fragment
atcgtacggcgggcagaccgggcagcagta

SEQ ID NO: 73 - DNA, 549 bp, Homo sapiens
  CDS 1..549: Endostatin
cactcgcaccgggacttccagccagtactccacctcgtagcactcaactcgccactctcgggaggaatgcggggaatccggggagcagacttccagtgcttccagcaggcacgggcagtaggactcgcaggaacattccgggcattcctctcgtcgcggctccaggacctctactcgatcgtacggcgggcagaccgggcagcagtaccaatcgtaaacctcaaggacgagctcctcttcccatcgtgggaggcactcttctcgggatcggagggaccactcaagccaggagcacggatcttctcgttcgacggaaaggacgtactccggcacccaacatggccacagaagtcggtatggcacggatcggacccaaacggacggcggctcacagagtcgtactgcgagacatggcggacagaggcaccatcggcaacaggacaggcatcgtcgctcctcggaggacggctcctcggacagtcggcagcatcgtgccaccacgcatacatcgtactctgcatcgagaactcgttcatgacagcatcgaag

SEQ ID NO: 74 - DNA, 69 bp, Artificial Sequence
  CDS 1..69: completely synthesized
tttgcttttgcttttaaagcttttaaaaaagcttttaaaaaatttaaaaaagcttttaaaaaagctttt

(19) United States
(12) Patent Application Publication
(10) Pub. No.: US 2012/0270770 A1
(43) Pub. Date: Oct. 25, 2012

(54) Anti-angiogenic peptides and their uses

(76) Inventor: Jesse Michael Jaynes, Auburn, AL (US)

(21) Appl. No.: 13/135,978

(22) Filed: Jul. 20, 2011

Related U.S. Application Data
(60) Provisional application No. 61/400,822, filed on Aug. 3, 2010.

(51) Int. Cl. A61K 38/08; C07K 7/08; A61P 9/10; A61P 25/00; A61P 17/06; A61K 38/10; A61P 19/02; A61P 1/00; A61P 27/16; A61P 37/06; A61P 9/12; A61P 29/00; C07K 7/06; A61P 35/00

(52) U.S. Cl. 514/1.9; 530/328; 530/326; 514/21.6; 514/17.9; 514/18.7; 514/19.3; 514/15.7; 514/13.3; 530/327

(57) ABSTRACT

This invention relates to novel synthetic lytic peptide fragments of full-length peptides with the capacity to modulate angiogenic activity in mammals. The invention also relates to the use of such peptides in pharmaceutical compositions and in methods for treating diseases or disorders that are associated with angiogenic activity.

This application claims priority under 35 U.S.C 119 (e) of U.S. Provisional The entire contents of the prior application U.S. Provisional are incorporated herein by reference.

### FIELD OF THE INVENTION

This invention relates to novel synthetic lytic peptide fragments of full-length peptides having the capacity to modulate angiogenic activity in mammals. The invention also relates to the use of such peptide fragments in pharmaceutical compositions and to methods for treating diseases or disorders that are associated with angiogenic activity.

### BACKGROUND OF THE INVENTION

Angiogenesis is a physiological process in which new blood vessels grow from pre-existing ones. This growth may be spontaneous formation of blood vessels or alternatively by the splitting of new blood vessels from existing ones.

Angiogenesis is a normal process in growth and development and in wound healing. It may play a key role in various healing processes among mammals. Among the various growth factors that influence angiogenesis naturally occurring vascular endothelial growth factor (VEGF) is known to be a major contributor by increasing the number of capillaries in a given network. VEGF is a signal protein produced by cells that stimulates angiogenesis. It is part of the system that restores the oxygen supply to tissues when blood circulation is inadequate. VEGF's normal function is to create new blood vessels during embryonic development, new blood vessels after injury, muscles following exercise, and new vessels to bypass blocked vessels

The process of angiogenesis may be a target for fighting diseases that are characterized by either under development of blood vessels or overdevelopment. The presence of blood vessels, where there should be none may affect the properties of a tissue and may cause for example, disease or failure. Alternatively, the absence of blood vessels may inhibit repair or essential functions of a particular tissue. Several diseases such as ischemic chronic wounds are the result of failure or insufficient blood vessel formation and may be treated by a local expansion of blood vessels. Other diseases, such as age-related macular degeneration may be stimulated by expansion of blood vessels in the eye, interfering with normal eye functions.

In 1971, J. Folkman published in the New England Journal of Medicine, a hypothesis that tumor growth is angiogenesis dependent. Folkman introduced the concept that tumor is probably secrete diffusable molecules that could stimulate the growth of new blood vessels toward the tumor and that the resulting tumor blood vessel growth could conceivably be prevented or interrupted by angiogenesis inhibitors

Tumor angiogenesis is the proliferation of a network of blood vessels that penetrates into cancerous growths supplying nutrients and oxygen while removing waste. The process actually starts with cancerous tumor cells releasing molecules that signal surrounding host tissue, thus activating the release of certain proteins, which encourage growth of new blood vessels. Angiogenesis inhibitors are drugs that block the development of new blood vessels, and. By blocking the development of new blood vessels. Researchers hope to cut off the tumor supply of oxygen and nutrients, which in turn might stop the tumor from growing and spreading to other parts of the body.

In the 1980s, the pharmaceutical industry applied these concepts in the treatment of disease by creating new therapeutic compounds for modulating new blood vessel in tumor growth. In 2004 Avastin (bevacizumab), a humanized anti-VEGF monoclonal antibody was the first angiogenesis inhibitor approved by the Food and Drug Administration for the treatment of colorectal cancer. It has been estimated that over 20,000 cancer patients worldwide have received experimental forms of anti-angiogenic therapy.

Angiogenesis represents an excellent therapeutic target for the treatment of cardiovascular disease. It is a potent, physiological process that underlies the natural manner in which our bodies respond to a diminution of blood supply to vital organs, namely the production of new collateral vessels to overcome the ischemic insult.

A decade of clinical testing, both gene and protein-based therapies designed to stimulate angiogenesis in under perfused tissues and organs has resulted in disappointing results; however, results from more recent studies with redesigned clinical protocols have given new hope that angiogenesis therapy will become a preferred treatment for sufferers of cardiovascular disease resulting from occluded or stenotic vessels.

### SUMMARY OF THE INVENTION

Because the modulation of angiogenesis has been shown to be a significant causative factor in the control of certain disorders and diseases, it is necessary to find agents which are safe and efficacious in either inhibiting or stimulating angiogenesis.

Additional features and advantages of the present invention will be set forth in part and in a description which follows, and in part will be apparent from the description, or may be learned by practice of the present invention. The objectives and advantages of the invention will be realized and attained by means of the elements, combinations, composition, and process particularly pointed out in the written description and appended claims.

To achieve the objects and in accordance with the purpose of the present invention, as embodied and broadly described herein, the present invention relates to new and novel synthetic lytic peptides which effectively enhance or inhibit angiogenesis and are therefore effective therapeutic agents in the treatment of disease in mammals.

In one aspect the present invention relates to synthetic lytic peptides having angiogenesis activity which are in the physical form of molecular fragments derived from corresponding full-length protein molecules. More particularly, this invention relates to peptide fragments that inhibit angiogenesis and are selected from peptide sequence:

(SEQ ID NO: 1)
FAKKFAKKFK,
(SEQ ID NO: 2)
IVRRADRAAVPIVNLKDELL
or
(SEQ ID NO: 3)
MFGNGKGYRGKRATTVTGTP.

In another embodiment of the present invention, a peptide fragment is provided having anti-inflammatory activity bearing the peptide sequence FAKKFAKKFK (SEQ ID NO: 1).

In another aspect, the present invention provides a method for treating chronic inflammation comprising administering to a mammal in need of such treatment a peptide fragment bearing the peptide sequence FAKKFAKKFK (SEQ ID NO: 1).

In yet another embodiment, the present invention provides a method for treating chronic inflammation related disorders or conditions selected from among arthritis, ulcerated colitis, Crohn's disease, cancer, multiple sclerosis, cervical spondylosis, tinnitus, systemic lupus, erythematosis, graft rejection, psoriasis, arteriosclerosis, hypertension and ischemia-reperfusion comprising administering to a mammal in need of such treatment a peptide fragment bearing the peptide sequence FAKKFAKKFK (SEQ ID NO: 1).

In another aspect, the present invention provides a pharmaceutical composition for the treatment of disorders or diseases which are ameliorated by the inhibition of angiogenisis comprising a peptide fragment having the sequence FAKKFAKKFK (SEQ ID NO: 1), IVRRADRAAVPIVNLKDELL(SEQ ID NO: 2), MFGNGKGYRGKRATTVTGTP(SEQ ID NO: 3) or combinations thereof.

In another embodiment of the present invention, a peptide fragment is provided according to claim 1, having the capacity to accelerate angiogenesis wherein said peptide fragment has the sequence FAKKFAKKFKKFAKFAFAF (SEQ ID NO: 4), FAKKFAKKFAKKFAK(SEQ ID NO: 6),KKFKKFAKKFAKFAF(SEQ ID NO: 7) or FAKKFAKKFKKF(SEQ ID NO: 8)or a pharmaceutically acceptable salt thereof.

In another aspect, the present invention provides a pharmaceutical composition for the treatment of disorders or diseases which are ameliorated by the acceleration of angiogenesis comprising treatment of a mammal with an effective amount of a peptide fragment having the sequence FAKKFAKKFKKFAKFAFAF (SEQ ID NO: 4), FAKKFAKKFAKKFAK(SEQ ID NO: 6), KKFKKFAKKFAKFAF(SEQ ID NO: 7) or FAKKFAKKFKKF(SEQ ID NO: 8) or a pharmaceutically acceptable salt thereof.

In another aspect, the present invention provides a method for treating ulcerative colitis in a mammal comprising administering to said mammal in need of such treatment an effective amount of the peptide as defined in claim 2 or a pharmaceutically acceptable salt thereof.

In another embodiment of the present invention, a peptide fragment is provided according to claim 2, having the capacity to modulate inflammatory bowel disease and ulcerative colitis in a mammal.

### BRIEF DESCRIPTION OF THE DRAWINGS

This patent application contains at least one drawing executed in color. Copies of this patent application publication with color drawing(s) will be provided by the Office upon request and payment of the necessary fee.

FIG. 1 illustrates the angiogenic process.

FIG. 2 provides physical characteristic of the 20 essential amino acids. The total volume, in cubic angstroms, is derived from the van der Waals' radii occupied by the amino acid when it is in a protein. Hydrophobicity is in kcal/mol and is the amount of energy necessary to place the amino acid, when in an alpha-helical protein, from the membrane interior to its exterior. Luminosity helps assigns the density of cyan (hydrophobic amino acids) or magenta (hydrophilic amino acids) to each glyph of the “molecular” font (Molly) that is described in this disclosure.

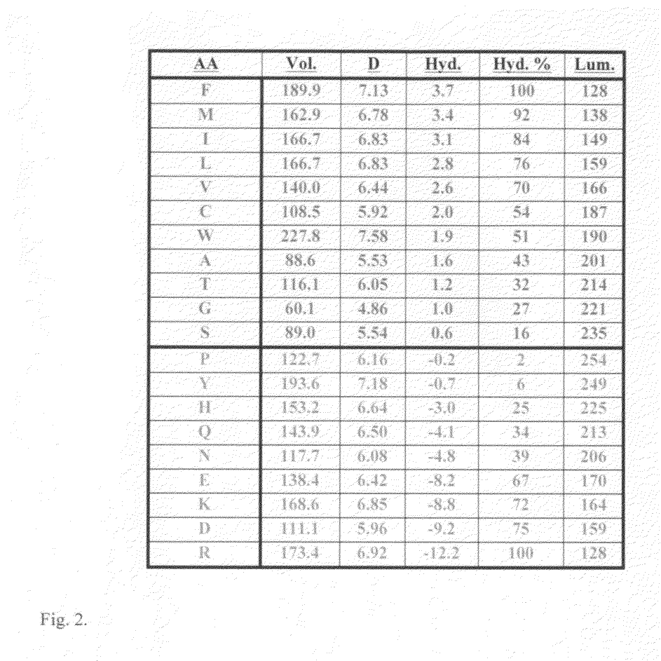

FIG. 3. Molly font wheel presented with single letter codes adjacent to each glyph. All hydrophobic amino acids are colored cyan while hydrophilic amino acids are magenta. The number values are relative hydrophobicities represented by the number of kcal/mole necessary to exteriorize an amino acid in an alpha helix from the inside of a lipid layer.

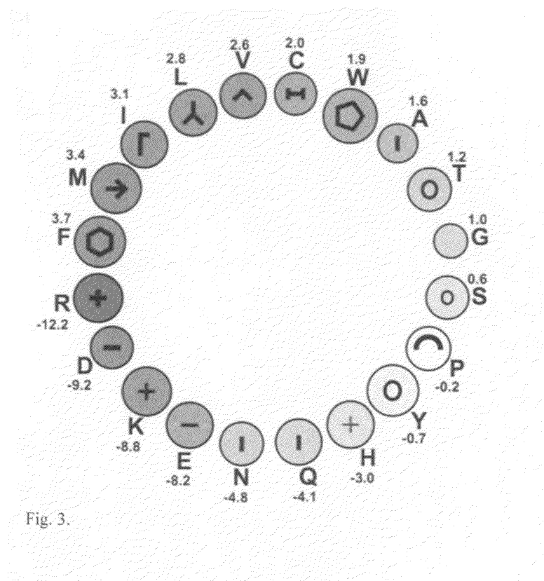

FIG. 4 illustrates three arrangements of naturally occurring peptides. The green band on the cylinders indicates the amino-terminus of the peptide while the gray band represents the carboxy-terminus. The cyan color represents regions that are predominately hydrophobic and the magenta color represents regions that are hydrophilic. Representative examples or natural peptides that fit this classification system are: mellitin-class 1; cecropins-class 2, and mangainins-class 3.

FIG. 5 shows sequences of natural lytic peptides melittin (SEQ ID NO: 9), Pipinin1 (SEQ ID NO: 10), adenoregulin (SEQ ID NO: 11), cecropin B (SEQ ID NO:12), adropin (SEQ ID NO:13), magainin 2(SEQ ID NO: 14) and their optimized analogs JC1A21 (SEQ ID NO: 74), JC15 (SEQ ID NO: 5) and JC3M1 (SEQ ID NO:16) along with color scale representation.

FIG. 6 shows sequences of a defensin (SEQ ID NO:17) and a protegrin (SEQ ID NO: 19) along with an optimized analog JC41 (SEQ ID NO:18). Color scale representation is included.

FIG. 7 shows the sequence of Human Plasminogen protein (SEQ ID NO: 20) including the sequence of angiostatin protein (SEQ ID NO: 21) derived from it (underlined sequence). PL 1 (SEQ ID NO: 22) and PL 2 (SEQ ID NO: 3) are also shown with shadowing.

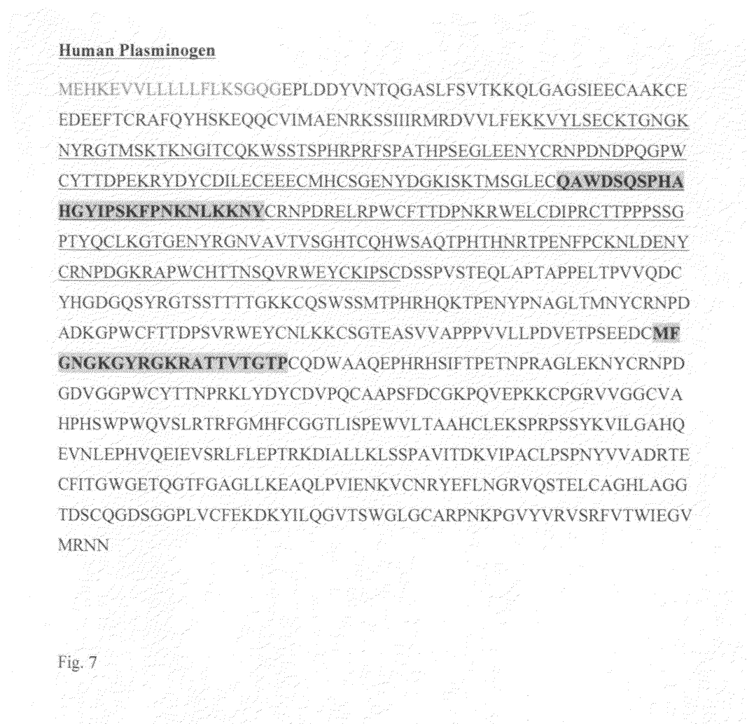

FIG. 8 shows sequences of fragments PL-1 (SEQ ID NO: 22) and PL-2 (SEQ ID NO: 3) derived from Human plasminogen protein. Color scale representation is included.

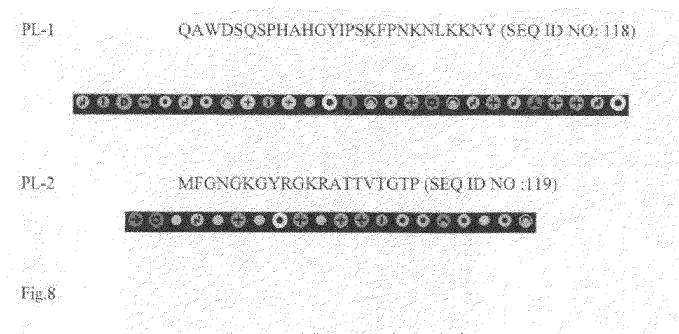

FIG. 9 shows the sequence of a fragment of Human Collagen XVIII (SEQ ID NO: 23). The underlined part of the sequence is the sequence of endostatin (SEQ ID NO: 73). Fragment C-1 (SEQ ID NO: 24) is shown with shadowing.

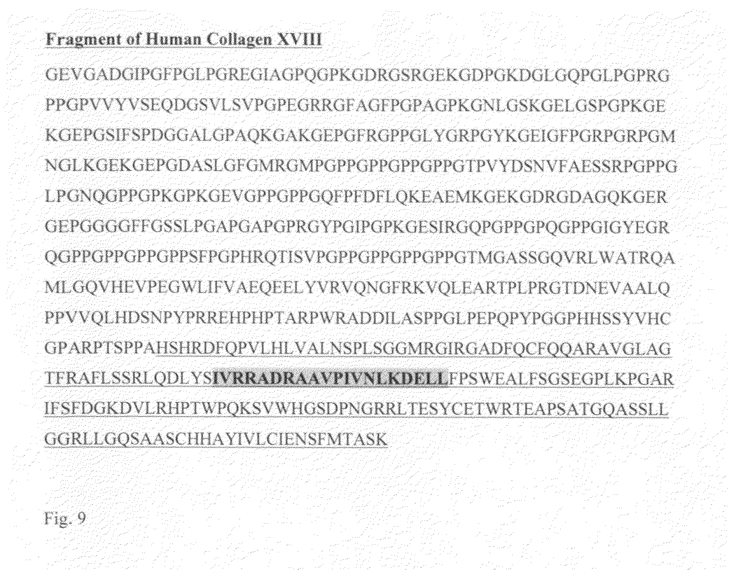

FIG. 10 shows sequence of the fragment C-1 (SEQ ID NO: 24) derived from Human Collagen XVIII. Color scale representation is included.

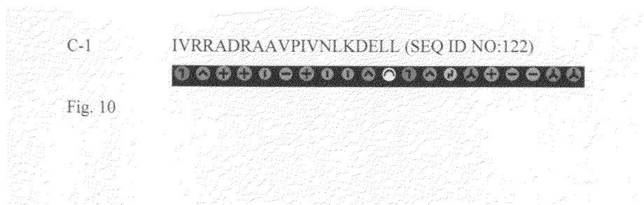

FIG. 11 shows the sequence of platelet factor-4 (SEQ ID NO: 25). Shadowed sequences represent PF1 (SEQ ID NO: 26) and PF2 (SEQ ID NO: 27).

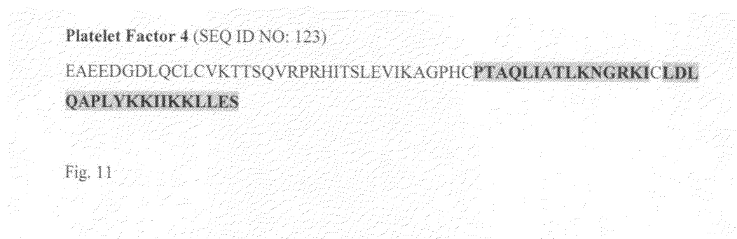

FIG. 12 shows sequences of fragments PF-1 (SEQ ID NO: 26) and PF-2 (SEQ ID NO: 27) derived form Platelet Factor 4. Color scale representation is included.

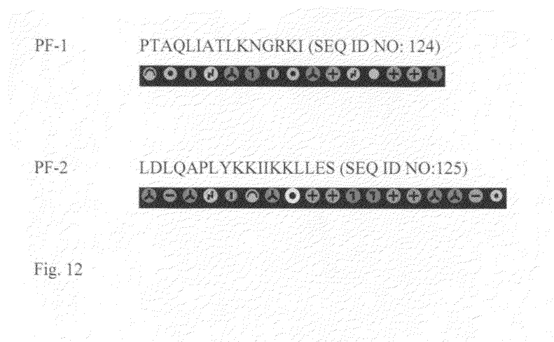

FIG. 13 illustrates Matrigel gels. A shows how a section of a Matrigel gel deposit looks like under the microscope soon after surgical implantation. The sample in B is derived from the control at the conclusion of the experiment. Intense activity is present with numerous cells attaching to the surface of the Matrigel. Cells begin to penetrate the deposit and organize into discrete structures that coalesce to form the beginning of tubes twisting and branching every way. In C, a typical sample from the peptide C-1 treatment is shown. This treatment caused far fewer cellular associations evident at the perimeter of the Matrigel deposit. Consequently, there were far fewer cells and cellular structures inside of the Matrigel. Only one peptide fragment from JC15, JC15-10N, possessed anti-angiogenic activity. A representative section of a Matrigel deposit from this set of animals is shown in D.

FIG. 14 shows anti-angiogenic activity of peptides of different lengths. As compared to control level the highest anti-angiogenic activity was obtained by peptides having less than 12 amino acids

FIG. 15 shows the sequences of natural and synthetic peptides of Example 6 in the color scale (Molly). The following sequences are shown: JC15 (SEQ ID NO: 5), JC15-18 (SEQ ID NO: 28), JC15-15C (SEQ ID NO: 29), JC15-10C (SEQ ID NO: 30), JC15-12N (SEQ ID NO: 8), JC15-10N (SEQ ID NO: 1) C-1 (SEQ ID NO: 24), PF-2 (SEQ ID NO: 27), PF-1 (SEQ ID NO: 26), PL-1 (SEQ ID NO: 22), PL-2 (SEQ ID NO: 3).

FIG. 16 illustrates the common motif of peptides of Example 6.

FIG. 17 shows the amino acid sequences of the chemokines of Table 7. The color scale is included and the sequences that are of interest are shadowed. Following chemokine sequences are shown: IL8 (SEQ ID NO: 31), MIG (SEQ ID NO: 32), IP-10 (SEQ ID NO: 33), MCP1 (SEQ ID NO: 34), MIP-la (SEQ ID NO: 35), RANTES (SEQ ID NO: 36).

FIG. 18. Comparison of an endostatin fragment with full-length D2A21 peptide and its generated fragments displayed by Molly

FIG. 19A is a display of selected fragments from several cytokines and endostatin (derivation on the left, designation on the right) compared to 10N of D2A21. The light brown background illustrates conservation of hydrophobicity and resultant amphipathy. The dark brown background indicates those amino acids that are out of place.

FIG. 19B displays the three-dimensional representations of the peptide fragments obtained using the UCSF Chimera software.

FIG. 20. Changes in the absolute CD4+ Cell Counts in the sera of Nasty the lion before and after peptide treatment.

FIG. 21 Changes in the absolute CD8+ Cell Counts in the sera of Nasty the lion before and after peptide treatment.

FIG. 22 shows the weight profile and CD4+/CD8+ ratios of FIV-infected lion before and after the peptide treatment. The CD4+/CD8+ ratios shown are absolute counts. The treatment consisted of weekly 70 mg I.M injections.

FIG. 23 shows X-ray figures of a normal ankle, an arthritic ankle and an arthritic ankle after several peptide treatments (10 mg subcutaneous injections once a week for one month and then one injection per month for maintenance).

FIG. 24 Statistical analysis of the IL-10 Knockout experiment colitis vs treatment

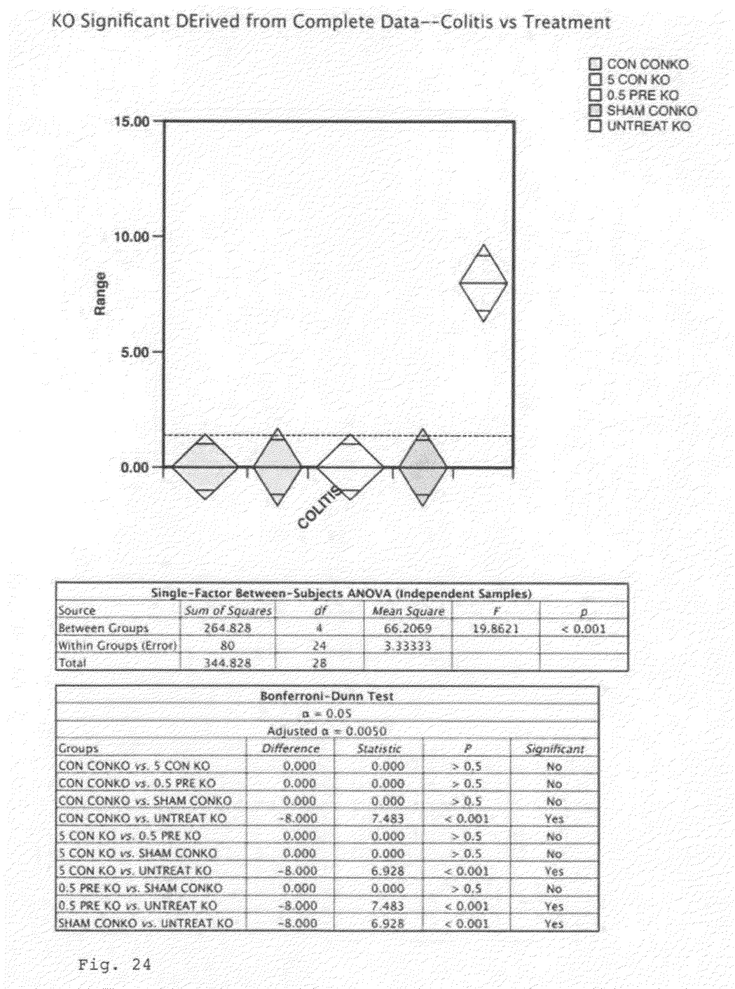

FIG. 25 Statistical analysis of the IL-10 Knockout experiment advanced disease vs treatment.

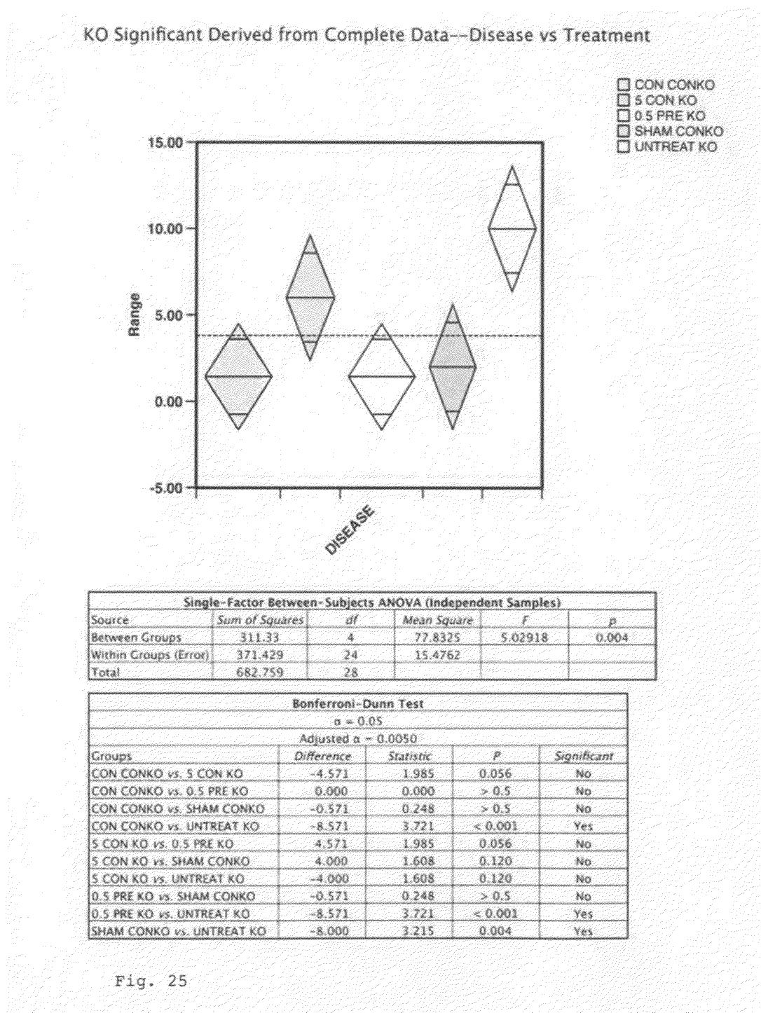

FIG. 26 Statistical analysis of the IL-10 Knockout experiment tumor development vs treatment

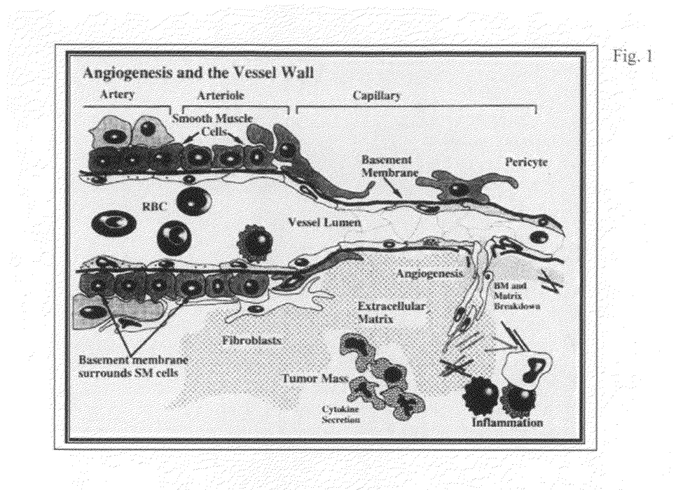

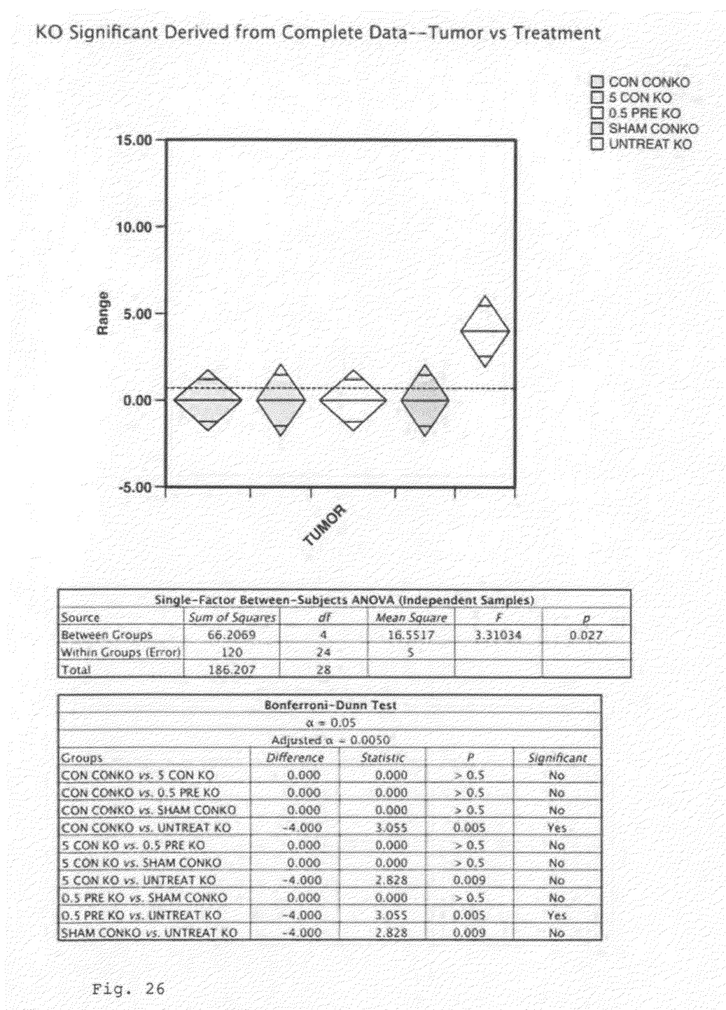

FIG. 1 illustrates the angiogenic process. Blood vessel walls in arteries, arterioles, and capillaries, are lined by basement membrane composed of endothelial cells. Angiogenesis occurs mainly in the capillaries or post-capillary venules. In response to cytokine stimulation, endothelial cells break down the basement membrane, migrate into the extra vascular space, proliferate, and reorganize to form a new vessel. The endothelial cell carries its own internal defense against stray growth factors and it is the most sensitive of all cells to growth control by cell shape. With recent research, it seems that mechanical forces on a cell are necessary for growth factors, cytokines and hormones, to function. These soluble molecules will remain inactive unless they are coupled to the mechanical forces generated by specific insoluble molecules (collagen and fibronectin). These insoluble molecules lie in the extra cellular matrix and bind to specific receptors and integrins on the cell surface. This allows a cell to pull against its extra cellular matrix and to generate tension over the interconnected cytoskeletal linkages. Thus, cell shape changes are a prerequisite for entry of that cell into the cell cycle and subsequent gene expression and cell division. In fact, for the endothelial cell it is not the area and shape configuration of the outer cell membrane that supplies the direct mechano-chemical information that permits DNA synthesis, but rather the shape of the nucleus. Nevertheless, nuclear shape is governed by the shape of the outer cell membrane and by tensile forces transmitted to the nucleus over the cytoskeletal network. When the shape of the nucleus is stretched beyond 60-70 microns there is net DNA synthesis.

The extra cellular matrix appears to contain special components; in particular, certain proteoglycans that bind and store these growth factors making them inaccessible to endothelial cells. For example, it is known that basic fibroblast growth factor (FGF) and vascular endothelial growth factor (VEGF) bind to heparin sulfate proteoglycan. The basement membrane itself may also inhibit endothelial growth. The laminin B1 chain contains two internal sites that, in the form of synthetic peptides having the sequence RGD and YSGR, inhibit angiogenesis. Furthermore, collagen XVIII is localized to the perivascular region of large and small vessels and a 187 amino acid fragment, called endostatin, is a potent and specific inhibitor of endothelial proliferation. Several other endogenous proteins block the multiplication of endothelial cells and exert a reduced angiogenic effect. In each case, the endothelial inhibitor activity is found in a fragment of a larger protein which itself lacks inhibitory activity.

Angiogenesis plays a role in various disease processes. It is well known that angiogenesis is involved in development of malignant tumors and cancer diseases. Moreover, angiogenesis is associated with rheumatoid arthritis. Chronic inflammation may also involve pathological angiogenesis; examples of angiogenesis related inflammation diseases are ulcerative colitis and Crohn's disease. Chronic inflammation has been implicated to be the primary causative factor in several diseases including arthritis, multiple sclerosis, cervical spondylosis, tinnitus, systemic lupus, erythematosis, graft rejection, psoriasis, atherosclerosis, hypertension, and ischemia-reperfusion. Lytic peptides are small proteins that are major components of the antimicrobial defense systems of numerous species (AMPs). They are a ubiquitous feature of nearly all multi-cellular and some single-cellular life forms. They generally consist of between 10-40 amino acids in length, which have the potential for forming discrete secondary structures. Often, they exhibit the property of amphipathy. An amphipathic a-helix may be depicted as a cylinder with one curved hemi-cylinder face composed primarily of non-polar amino acids while the other face is composed of polar amino acids

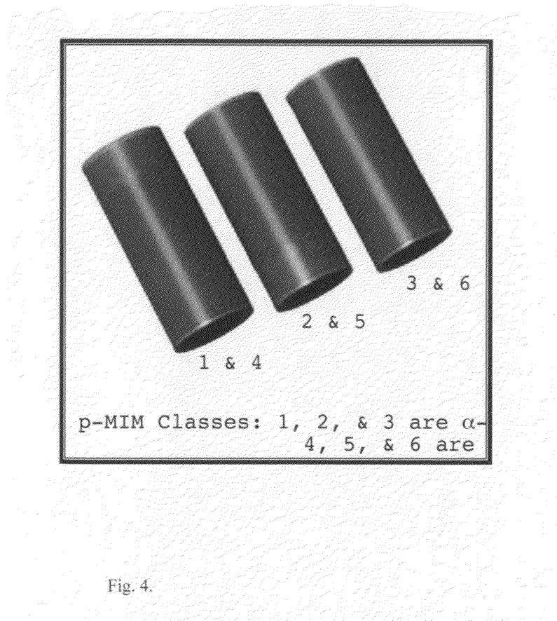

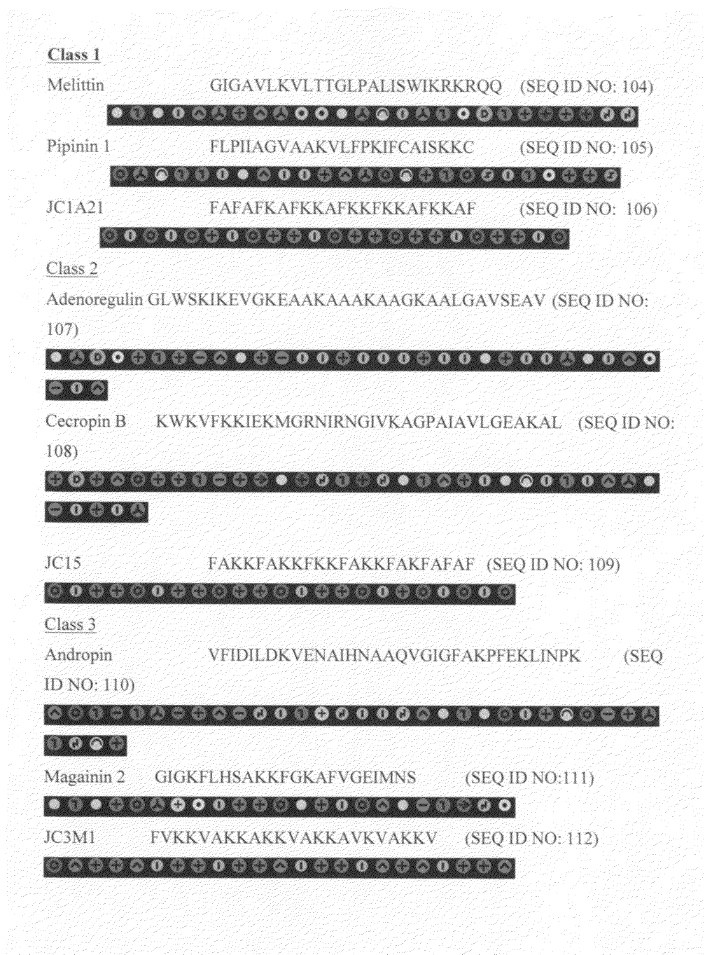

Four distinct types of lytic peptides were discovered in the last decade; examples of each type are melittin, cecropins, magainins, and defensins. The properties of naturally occurring peptides suggest at least three distinct alpha-helical classes consisting of different arrangements of amphipathic and hydrophobic regions (FIG. 4). The green band on the cylinders indicates the amino-terminus of the peptide while the gray band represents the carboxy-terminus. The cyan color represents regions that are predominately hydrophobic and the magenta color represent regions that are hydrophilic. Representative examples of natural peptides, which fit this classification system are: melittin-class 1, cecropins-class 2, and magainins-class 3 (note, 99% of all the known natural peptides fall within this classification system, data not shown). Therefore, separate synthetic peptides can be subdivided into distinct classes based on what has been observed in Nature. Some examples of natural lytic peptides and their sequence as cast in the glyph motif are listed in FIG. 5, along with representative optimized analogs. These are shown in a typical linear array and are read from left to right.

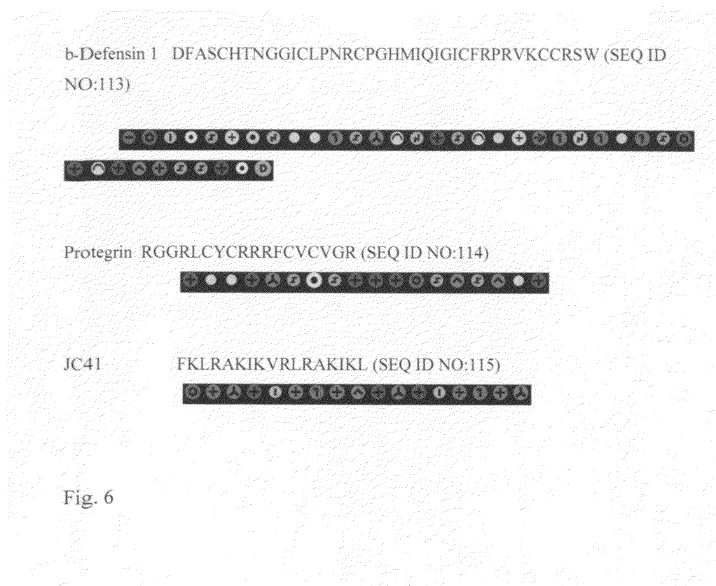

The only natural lytic peptides that assume a b-conformation are the defensins and protegrins. They can assume this shape because of intra-disulfide linkages that lock them into this form, an absolute requisite for activity. We have completely novel classes of peptides that form b-sheets without the necessity of disulfide linkages. An example, JC41 is shown in FIG. 6. The columnar array of hydrophobic and positive charged amino acids is apparent when the peptide adopts an amphipathic b-form. However, the width of the columns is narrower but overall length is greater than a peptide that adopts an amphipathic a-helix conformation.

### Anti-Angiogenesis

Lytic peptides are active in eliminating tumor-derived cells by causing direct osmotic lysis. Based on this demonstrable activity, reason suggests that in order to demonstrate in vivo activity the peptide must be injected directly into the tumor. Indeed, that is the case. With just a few injections over a period of several days, tumors are permanently eliminated using the most active anti-tumor peptide, D2A21, yet tested. A follow-up series of experiments was designed to determine what occurs when this peptide is injected in a site removed from the tumor (in other words, can it express any systemic activity)?” The results were unexpected as most of the tumors also disappeared in several animal tumor models. However, in some cases there was little activity. To determine what might be happening in vivo, radiolabeled D2A21 was chemically synthesized with all alanines labeled with either ³H or ¹⁴C. Since the labeling pattern was asymmetric, it enabled us to follow the physical state of the peptide once it had been injected into the animal by comparing the unique ratios of ³H/¹⁴C that would result if the peptide experienced proteolysis. It was found that within minutes the labeled peptide was hydrolyzed to fragments of various lengths no matter the route of administration but in the circulation approximately 14% of the radiolabel persisted for at least 24 hours with minimal further degradation (unpublished observations). The possibility emerged that the systemic in vivo anti-cancer activity was retained within specific fragments of D2A21. Based on these results, several peptide fragments were selected for further study as outlined herein below.

### DETAILED DESCRIPTION OF THE INVENTION

Selected Peptide Fragments From Full-length Corresponding Protein The synthetic peptide fragments of the present invention are listed in Table 1 Fragments PL-1 (SEQ ID NO: 22) and PL-2 (SEQ ID NO: 3) are peptide fragments of plasminogen protein (SEQ ID NO: 20). Fragment C-1 (SEQ ID NO: 2) is a peptide fragment of the larger protein molecule Collagen XVIII (SEQ ID NO: 23) endostatin fragment (SEQ ID NO: 73). The two peptides PF-1 (SEQ ID NO: 26) and PF-2 (SEQ ID NO: 27) are fragments of platelet factor-4. Included are also several fragments of JC15, JC15-18N, JC15-12N, JC15-15C, JC15-10C, JC15-10N,

The peptide fragments of the present invention were prepared by the method Fmoc peptide synthesis procedure that is a typical method for the preparation of peptide sequences.

### Procedure for Determining Angiogenic Activity of Peptide Fragments (Either Acceleration or Inhibition)

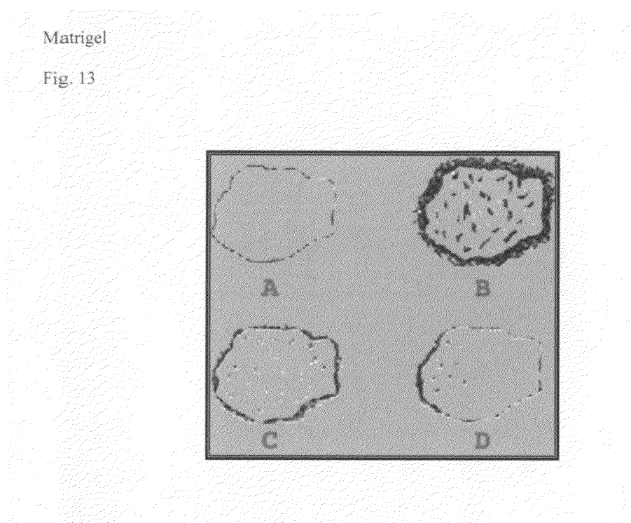

Matrigel deposits were surgically implanted on both sides of 4 mice per treatment yielding a possible 8 samples per treatment. Matrigel is a polymeric substance that appears to be relatively inert in animals and it can serve as a matrix that allows experimentation in vivo on many different difficult-to-study-processes. Prior to implantation, the Matrigel was allowed to imbibe fibroblast growth factor 1 (FGF1). This protein is a powerful inducer of angiogenesis and its presence guarantees that sufficient activity will be observed within the allotted time period of the experiment. Therefore, any inhibition of angiogenesis is likely to be a real phenomenon as the experiment has been set to heavily favor the angiogenic process. Angiogenesis occurs by day 14 and beginning Day 1 (Day 0=day of implantation), mice were injected IP daily with 20 μg of peptide in 100 μl of normal saline. The animals were sacrificed on Day 14, and each Matrigel deposit divided longitudinally and fixed in 10% buffered formalin. One of the halves of each Matrigel deposit was then sectioned. Read-out for this experiment was via histology, with semi-quantitative/qualitative counting of migration of cells and their subsequent assembly of lumenal structures within the Matrigel. This method allows us to observe the full physiologic spectrum of effects, and was useful in delineating trends. FIG. 13 shows the summary rendition of what, on average was observed. In FIG. 13, A represents what a section of a Matrigel gel deposit looks like under the microscope soon after surgical implantation. The sample in B is derived from the control at the conclusion of the experiment. Intense activity is present with numerous cells attaching to the surface of the Matrigel. Cells begin to penetrate the deposit and organize into discrete structures that coalesce to form the beginning of tubes twisting and branching in many directions. These venules eventually connect with the system carrying blood and it is possible to see red cells and lymphocytes within them. This process is called “arborization”, derived from the fact that the angiogenic process most closely resembles the growth of roots and branches of trees. All but two of the peptide treatments looked, more or less, like B (all the peptides of Table 1 were tested). In C, a typical sample from the peptide C-1 treatment is shown. This treatment caused far fewer cellular associations evident at the perimeter of the Matrigel deposit. Consequently, there were far fewer cells and cellular structures inside of the Matrigel. Only one peptide fragment from JC15, JC15-10N (SEQ ID NO: 1), possessed anti-angiogenic activity. A representative section of a Matrigel deposit from this set of animals can be found in D. Importantly and surprisingly, not the numbers of internal cells and structures within the Matrigel deposit were reduced, but there was a seeming asymmetry of their organization where activity was evident. There were large regions of Matrigel that had no visibly associated structures and few single cells, including on the periphery, while other regions had some limited activity. This experiment demonstrates that portions of endostatin and JC15 possess significant anti-angiogenic activity.

Table 2 shows the data collected from the experiment using semi-quantitative/qualitative scale for measuring angiogenic activity. This method is used as an initial assessment to find compounds that possess angiogenic activity, molecules that either accelerate or inhibit the process. This system ranks each sample using a 0 to 4 plus (+) scale. Thus, B in FIG. 13 would yield a score of +++ while no + sign would yield a value of 0, as in A in FIG. 13. In Table 3 the data is modified to a numerical form and plotted averages are shown.

Analysis shows that significant differences exist between the JC15-10N & C-1 pair, from the rest of the treatments. However, JC15-10N (SEQ ID NO: 1) and C-1 (SEQ ID NO: 24) are not significantly different from one another.

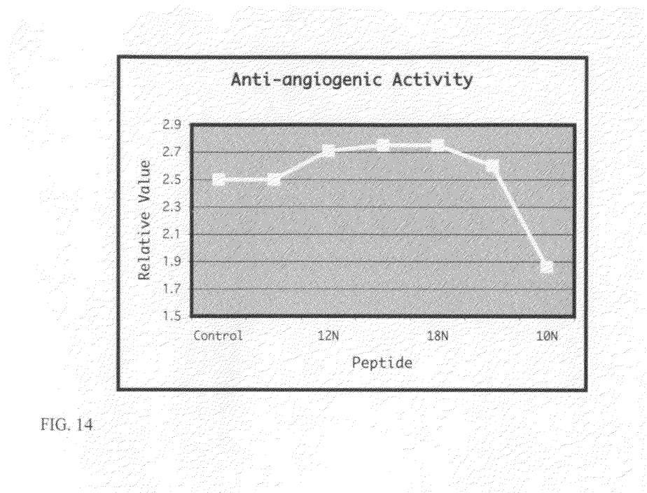

Based upon these data. It would appear that several of the peptides may actually promote angiogenesis. For example, mice treated with JC15-18N, JC15-15C, and JC15-12N, all show levels of activity higher than the control. Indeed, Matrigel deposits treated with the latter two peptides had the only top level (++++) scores of the entire experiment. a-amphipathic peptides of high positive charge density can cause cell proliferation, with the effect being more pronounced in peptides below 18 amino acids in length. These smaller peptides' lytic activity is greatly reduced because they are simply too short to physically span the membrane, the site of their direct mode of action. It is interesting that the level of activity is closer to the control in the full-length JC15 treatment group as opposed to some of its smaller fragments. A peak of angiogenic activity above the control is seen when the peptide is between 12 and 18 amino acids in length, culminating in observable anti-angiogenic activity when the peptide is shorter than 12 amino acids (FIG. 14).

### EXAMPLE 6

### Structure/Function Relationships of the Peptides and Their Anti-Angiogenesis Effect

Table 3 allows one to see similarities or differences in the presence or absence of charged amino acids and their position with respect to hydrophobic (white rectangles) and other hydrophilic amino acids (dark rectangles) in the peptides tested in the Matrigel experiment.

One can see structural similarities, within sequence motifs, when sequences are presented as in Table 3. Of course, all of the fragments of JC15 are going to be identical to different regions of the full-length JC15 molecule. However, it is also apparent that the endostatin fragment, C-1, has more than just a passing resemblance to JC15 and its fragments, as do portions of the peptides from plasminogen. In addition, the C-terminal half of PF-2, derived from platelet factor 4, shares similarly significant structural homology.

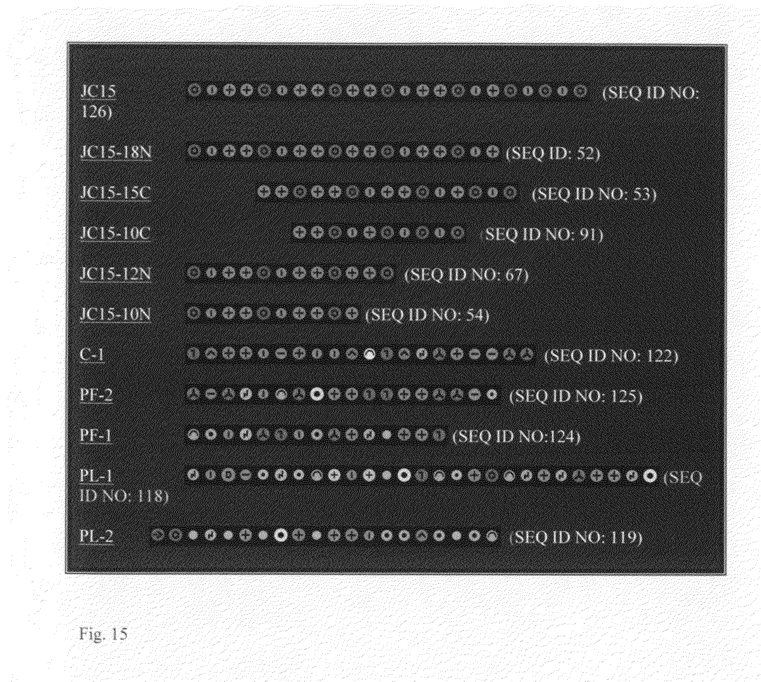

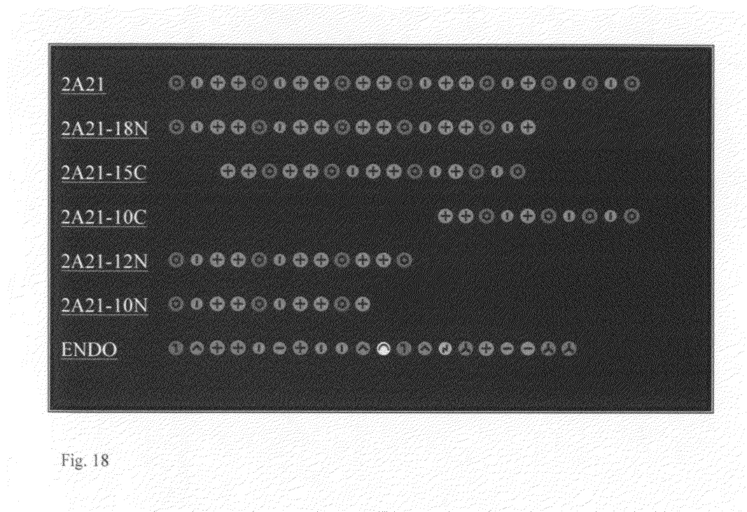

As can be seen from FIGS. 15 and 18, there is a close physico-chemical relatedness of C1 (EndoF) and JC1510N (D2A21-10N) when illustrated with Molly.

Are these structurally homologous regions enough alike to all modulate angiogenesis in some way? Most biochemical processes occur at the surfaces of different macromolecules that associate or bind to specific regions on one another within a discrete three-dimensional space. These binding sequences are often rather short stretches of a protein, say, 4 to 8 amino acids. It is entirely within the realm of possibility that there are only 5 or so amino acids that comprise the critical binding region that interacts specifically with target macromolecules initiating an in vivo anti-angiogenic response. The data support the hypothesis that C-1 and JC15-10N possess this binding region.

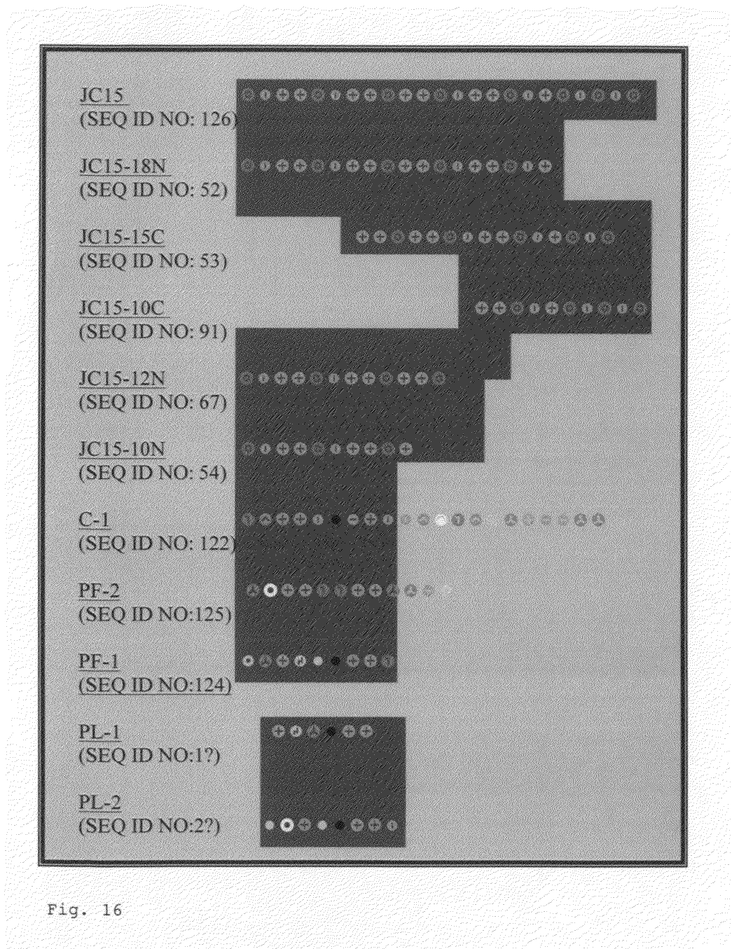

In FIG. 15 the sequences are casted in Molly and FIG. 16 is a simple schematic illustration derived from FIG. 15. By keeping in mind that each magenta square is, with just a few exceptions, a “+” charged amino acid, the following conclusions can be made:
JC15 and all of its fragments possess the same type of internal sequence of 7 or 9 amino acids, with JC15 and JC15-18N retaining one of each. Noting the shift of one amino acid, most importantly, the same can be said for the peptides C-1, *PF-1, and *PF-2.
The anti-angiogenic fragment must be of a certain length. Even if a fragment retains the putative 7 or 9 amino acid binding sequence, like JC15, JC15-18N, JC15-15C, and JC15-12N, it still cannot exert an anti-angiogenic effect. Clearly, the simplest explanation is that these sequences cannot “fit” into the target-binding site. How critical this size requirement is, can be borne out by the fact that a fragment identical to JC15-10N, but with the addition of two amino acids, JC15-12N, does not inhibit angiogenesis. In fact, it may actually cause an opposite effect. Then, one may ask, why does C-1 possess anti-angiogenic activity when it seems to violate the size requirement, after all, it is 21 amino acids in length? My best guess, at this time, is that the proline, with just 2 amino acids separating it from the putative binding sequence, directs the rest of the fragment away from the target-binding site, reducing interference to a minimum. After all, that is proline's function—to allow bends and turns in proteins. Alternatively, it could be processed in the animal to a shorter fragment.
More than a specific length is necessary. JC15-10C and JC15-10N are the same size yet JC15-10N is the only one that possesses anti-angiogenic activity. Even though JC15-10C contains a probable 7 amino acid binding sequence, the addition of 3 hydrophobic amino acids on the C-terminal end of JC15-10C are enough to negate binding, 2 of the 3 being bulky phenylalanines. In addition, one can conclude that a more optimal binding fragment contains several pairs of charged or other hydrophilic amino acids in the binding sequence see JC15-10N and C-1. Perhaps, another reason why JC15-10C was inactive.
The “interchangeability” of like amino acids is most apparent in comparison of JC15-10N with C-1. Even though their sequences are quite different, almost perfect correspondence is observed when they are cast in the molecular font. It is possible that JC15-10N could be made even more active by removing one of the internal hydrophobic amino acids and reducing its length by one or two amino acids from its C-terminal end. Also, the addition of a negatively charged amino acid, within the charged pair, may be desirable.

### EXAMPLE 7

### Chemokine Anatomy and the Design of Novel Domains to Delineate Specific Cellular Activities

Chronic inflammation has been implicated to be the primary causative factor in various diseases including: arthritis, multiple sclerosis, cervical spondylosis, tinnitus, systemic lupus, erythematosis, graft rejection, psoriasis, atherosclerosis, hypertension, and ischemia-reperfusion. The surprising fact is that just a handful of pro-inflammatory chemokines are responsible and according to this disclosure JC15-10N has structural analogies within the sequences of each molecule.

While there are more than 50 chemokines that have been characterized, but a clearly smaller set is involved in diseases. Table 4 provides internal sequence of a number of chemokines and Table 5 shows the chemokines involved in several diseases.

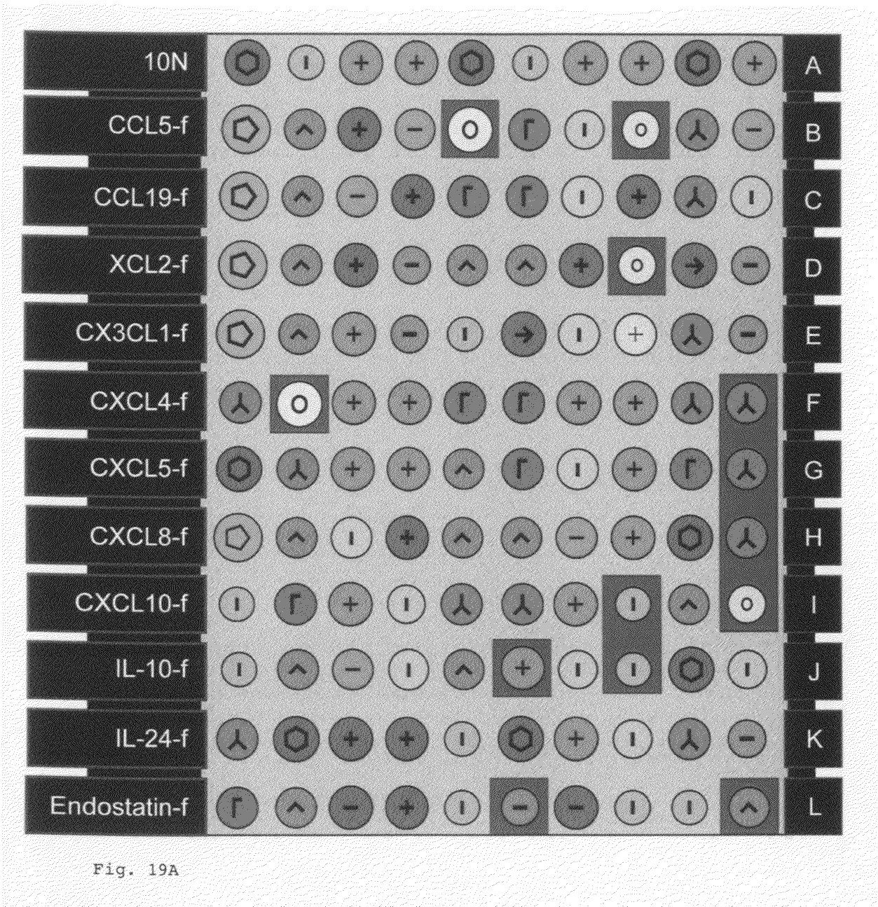

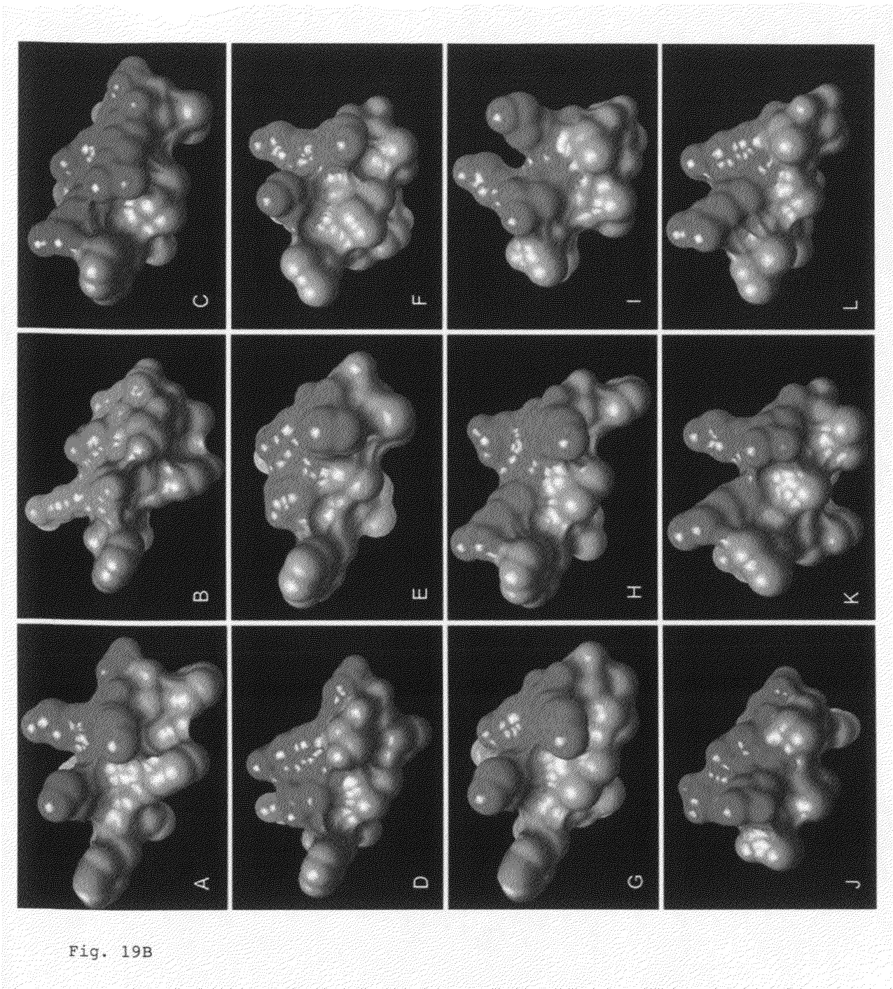

The chemical/structural similarities of the chemokines in FIG. 19 A with JC15-10N are easy to recognize. They conserve amphipathy and charge density to a high degree and their 3-dimensional structure (FIG. 19B) would be quite similar to JC10. Mostly they all appear after a proline and are more often than not at the C-terminus—this yields distinct domains. Also, it is interesting that the internal sequence of WVQ has been conserved with the divergent one IP-10 possessing AIK that conserves hydrophobicity exactly. I would predict that all of the above sequences would possess anti-angiogenic and anti-inflammatory activity much like JC15-10N. Thus, these key sequences of each domain, within the specific protein, no doubt functions as a down-regulator or off/brake switch for the inflammatory process.

### EXAMPLE 8

### Antiangiogenic and Anti-Inflammatory Effects of JC15-10N as Tested in a Lion Infected with FIV

Nasty is a male lion in North Carolina Zoological Park. He was diagnosed to suffer Feline Immunodeficiency virus FIV. FIV attacks the immune system of cats, much like the human immunodeficiency virus (HIV) attacks the immune system of human beings. FIV infects many cell types in its host, including CD4+ and CD8+ T lymphocytes, B lymphocytes, and macrophages. FIV eventually leads to debilitation of the immune system in its feline hosts by the infection and exhaustion of T-helper (CD4+) cells.

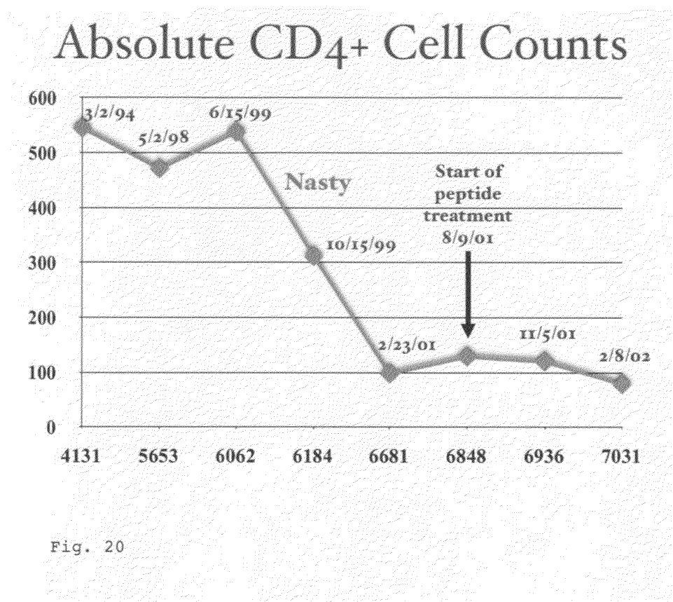

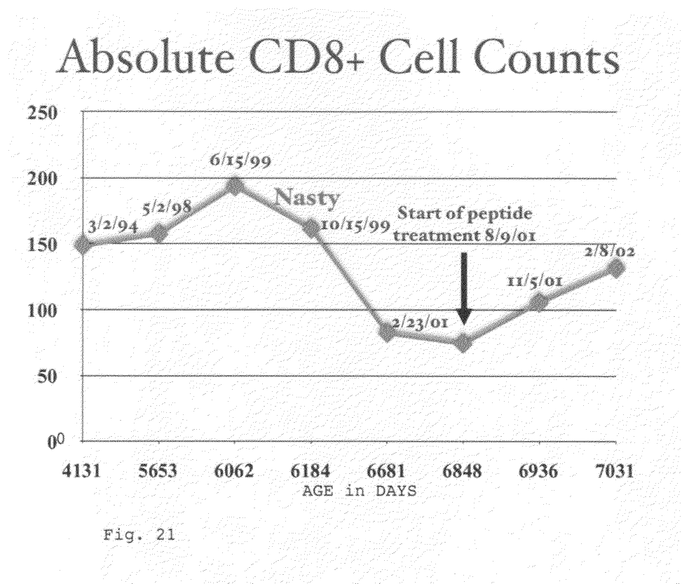

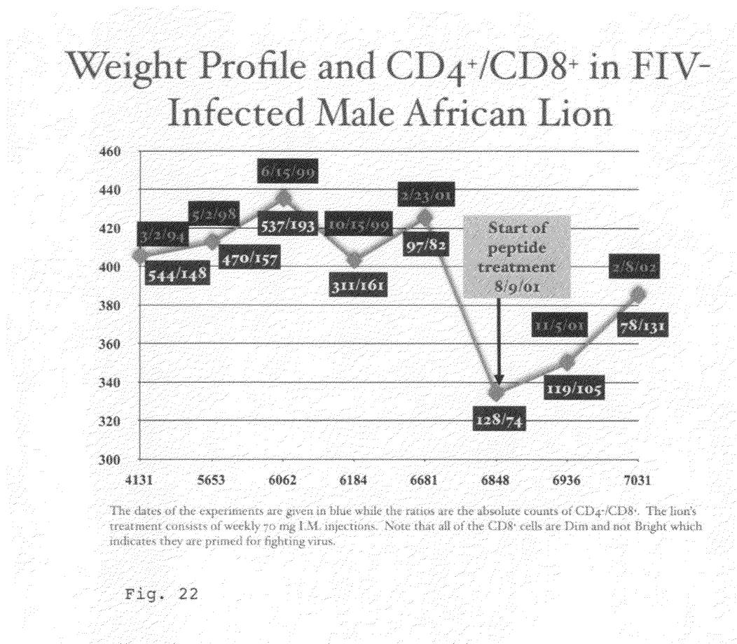

Nasty was treated weekly with 70 mg I.M injections of JC15-10N. FIG. 20 shows changes in the absolute CD4+ Cell Counts of Nasty before and after peptide treatment. It can be seen that starting of peptide treatment stabilized the CD4+ cell counts. FIG. 21 shows changes in the absolute CD8+ cell counts of Nasty before and after peptide treatment. Starting of the treatment prevented the decrease and actually, the cell counts began to rise soon after the treatment. FIG. 22 shows weight profile of Nasty before and after the peptide treatment along with changes in CD4+/CD8+ ratio. As can be seen, the weight of the lion began to rise immediately after beginning of the peptide treatment.

### EXAMPLE 9

### Treatment of Pancreatic Cancer with JC15-10N

A 73 year old woman diabetic since 12/01 was diagnosed with Stage IV pancreatic cancer in May 2002 with metastatic diseases in her liver. Median survival time of patients with Stage IV pancreatic cancer is 4.5 months. Median survival time of patients with Stage IV pancreatic cancer when treated with gemcitabine is 4.8 months. The longest anyone lived on gemcitabine treatment has been 19 months.

The patient of this case started JC15-10N peptide treatment on August 2002. The patient was given 0.5 mg/kg peptide sub-cutaneously. The patient weighted 128 lbs. and therefore she received 29 mg/injection per week. The disease in her liver diminished significantly. The patient required no more diabetic medication, which indicated that her primary tumor was regressing. After receiving the peptide for more than 19 months, the patient was doing fine. The patient passed away from unrelated causes on July 2004.

### EXAMPLE 10

### Treatment of arthritis with JC15-10N peptide

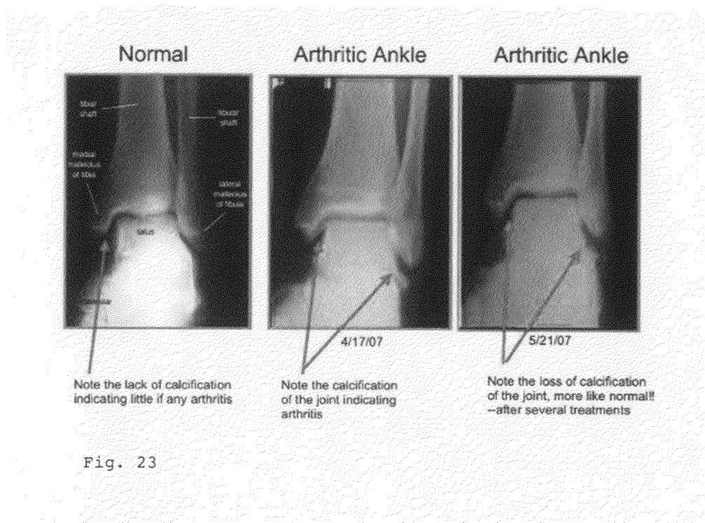

A patient with arthritis was treated by subcutaneous injection of 10 mg once a week for one month and then once a month for maintenance doses. A visible indication of arthritis is calcification of joints. The calcification of the ankle joints disappeared during this time indicated in the x-ray results are shown in FIG. 23.

### Efficacy of Novel Anti-Inflammatory Compounds in a Murine Model of Ulcerative Colitis: IL-10 Deficient Mice

Interleukin 10 (IL-10) is known for its anti-inflammatory properties in mammals. Several cell types including monocytes and lymphocytes produce it. It has been shown to down-regulate Th 1 cytokines, MHC class II antigens and co-stimulatory molecules on macrophages. There is good evidence that it also acts as an immuno-regulator in the intestinal tract and plays a positive role in limiting inflammatory bowel disease in humans. Clinical research has demonstrated that patients with inflammatory bowel disease, ulcerative colitis and Crohn's disease are predisposed to cancers of the intestinal tract.

An IL-10 deficient strain of mouse was used in the study to determine the ability of JC15-10N (a novel anti-inflammatory molecule) to limit their developing IBD and subsequent colon cancer.

The Wild Type and IL-10 deficient mouse strains used in this study was obtained from Jackson Laboratories and are designated as:

129SvEv Wild Type

129SVEV-IL10^{-/-}

The following groups of animals comprised the present experiment:

Controls:
1. Controls (129 SvEv 129^{-/-} untreated)
2. Controls (129 SvEv 129^{-/-} Sham/Saline Only)
3. 129 SvEv Wild-type Controls (untreated)
4. 129 SvEv Wild-type (Sham/Saline Only)

Treatments with JC15-10N:
1. Pre-inflammatory (129 SvEv 129^{-/-}-Prevention-1 injection/week)
2. Frank inflammatory (129 SvEv 129^{-/-}-Treatment-1 injection/week)
3. Pre-inflammatory (129 SvEv 129^{-/-}-Prevention-1* injections/week)
4. Frank-inflammatory (129 SvEv 129^{-/-}-Treatment-1* injections/week)
5. 129 SvEv Wild-type (Prevention-1 injection/week)
6. 129 SvEv Wild-type (Treatment-1 injection/week)
7. 129 SvEv Wild-type (Prevention-1* injections/week)
8. 129 SvEv Wild-type (Treatment-1* injections/week)
9 animals/group X 12 groups (including controls)=108 mice×2 repetitions=216 mice; 1 injection/week=0.5 mg/kg subQ and 1* injection/week=5.0 mg/kg subQ injection/week.
In order to encourage inflammation, prior to the start of the experiment, pathogenic murine strains of E. coli and E. faecalis bacteria were administered to the mice in the appropriate treatment groups via oral and anal gavages, designated EC/EF.

### Experimental Objectives

### Objective 1: To Test the Anti-Inflammatory Properties of JC15-10N.

Pre-inflammatory (4 wks.) and frank-inflammatory animals (6 wks.-8 wks) were treated with JC15-10N via subcutaneous injections every week for 14 weeks. The compound was diluted in saline to obtain treatment doses of 0.5 and 5.0 mg/kg of body weight respectively and was delivered subcutaneously using tuberculin needles. As controls, some animals were injected with saline only or remained uninjected during the course of the experiment. The procedure described by Hem and coworkers (Laboratory Animals Ltd. 1998. V 32. 364-368) was used to collect 100 micro-liters of blood from the lateral saphenous vein of all test animals once per week over the 14 weeks of peptide therapy. Blood serum was analyzed for changes in the levels of pro-inflammatory and anti-inflammatory cytokines over the 14-week treatment period. At the end of the 14-week treatment period animals were euthanized using a CO₂ chamber and flushing the peritoneum with PBS will collect peritoneal lavage fluid. Colons were removed and flushed separately with PBS. Cytokine levels in the peritoneal lavage fluids and colonic fluids of all animals were compared. Following colonic flushes; colons were splayed and examined for inflammatory lesions. Lesions were excised with scissors and the remainder of the colon rolled into gut rolls. Both lesions and gut rolls were fixed by incubation for 24 hrs. In 10% formalin, rinsed in 70% ethanol and held in PBS until they were embedded and prepared for histological analyses.

### Objective 2: To Determine the Effects of JC15-10N Therapy on Immune System Stimulation.

Immune system cells are major sources of inflammatory cytokines. The impact of treatments on the development and release of lymphocytes from primary and secondary lymphoid organs was analyzed. The influence of treatments on the numbers of circulating immune system cells including macrophages, neutrophils, dendritic cells and lymphocytes was monitored. Immune cells were isolated from the blood and peritoneal lavage fluid collected and their numbers quantitated. Additionally, primary (thymus and bone marrow) and secondary (spleen and lymph nodes) lymphoid tissues were removed from all animals at the time of sacrifice for comparative histological analyses. The sera and tissue samples of both are in the process of being analyzed.

### Results

Preliminary data analyses demonstrate a profound protective effect of JC15-10N at both dosages (with 0.5 mg/kg being somewhat better than the 5.0 mg/kg dosage).
There was significant reduction of colitis, presence of advanced disease and subsequent development of colon cancer in JC15-10N-treated IL-10^{-/-} mice compared to the control IL-10^{-/-} receiving no treatment.
The wild-type mice showed no disease symptoms across all treatments.

### Explanation of the Statistical Analyses Shown Below:

The most important results were abstracted from the total analysis.
Single-Factor Between-Subjects ANOVA (independent samples) and a further Bonferroni-Dunn test are shown.
The legend designations are:
   CON CONKO=IL-10^{-/-} mice, no exposure to EC/EF and receiving no treatment
   5 CON KO=IL-10^{-/-} mice, exposure to EC/EF and receiving Frank 5.0 mg/kg JC15-10N treatment
   0.5 PRE KO=IL-10^{-/-} mice, exposure to EC/EF and receiving Preventative 0.5 mg/kg JC15-10N treatment
   SHAM CONKO =IL-10^{-/-} mice, no exposure to EC/EF and receiving saline treatment
   UNTREAT KO=IL-10^{-/-} mice, exposure to EC/EF and receiving no treatment
The data shown is comprised of
   Colitis vs Treatment
   Advanced Disease vs Treatment
   Tumor development vs Treatment

### EXAMPLE 12

### Ability of 10 N to Inhibit Migration of Endothelial Cells

The results in Table 6 are gathered from an in vitro experiment and show the ability of JC 15 10N to inhibit the migration of endothelial cells (the “minus” values). This migration is a requisite step in the formation of new blood vessels. The fact that 10N retards this activity demonstrates its ability to block angiogensis and hence explains, at least partially, its inhibitory effect on cancer. The shading indicates the presence of VEGF (vegetative endothelial growth factor). This growth factor causes endothelial cell migration and consequent assembly of blood vessels. Because 10N inhibits this migration even in the presence of VEGF (gray shading) is highly significant.

Cell Migration Assay

Cell migration was performed as previously described. In brief, a HMEC-1 monolayer was scraped making a 1-mm wide denuded area then stimulated with VEGF and 10N and the area unoccupied by the migrating cells was determined using MetaMorph and expressed as a percentage of control.

### Tables

TABLE 1
Lytic Peptide Fragments of the Present Invention

| Name | Sequence | # | MWT | *MWT |
|---|---|---|---|---|
| JC15 | FAKKFAKKFKKFAKKFAKFAFAF | 23 | 2775.48 | 3388.48 |
| | (SEQ ID NO: 5) | | | |
| JC15-18N | FAKKFAKKFKKFAKKFAK (SEQ | 18 | 2191.79 | 2804.79 |
| | ID NO: 28) | | | |
| JC15-12N | FAKKFAKKFKKF (SEQ ID | 12 | 1517.93 | 1953.93 |
| | NO: 8) | | | |
| JC15-15C | KKFKKFAKKFAKFAF | 15 | 1864.36 | 2359.36 |
| | (SEQ ID NO: 7) | | | |
| JC15-10C | FAKKFAKFAF (SEQ ID NO: | 10 | 1204.48 | 1463.48 |
| | 30) | | | |
| JC15-10N | FAKKFAKKFK (SEQ ID NO: 1) | 10 | 1242.58 | 1619.58 |
| PL-1 | QAWDSQSPHAHGYIPSKFPNKNL | 27 | 3156.52 | 3615.52 |
| | KKNY (SEQ ID NO: 22) | | | |
| PL-2 | MFGNGKGYRGKRATTVTGTP | 20 | 2099.41 | 2417.41 |
| | (SEQ ID NO: 3) | | | |
| C-1 | IVRRADRAAVPIVNLKDELL (SEQ | 20 | 2261.70 | 2648.70 |
| | ID NO: 24) | | | |
| PF-1 | PTAQLIATLKNGRKI (SEQ ID | 15 | 1623.97 | 1882.97 |
| | NO: 26) | | | |
| PF-2 | LDLQAPLYKKIIKKLLES (SEQ | 18 | 2113.62 | 2477.62 |
| | ID NO: 27) | | | |
| D4E1 | FKLRAKIKVRLRAKIKL (SEQ ID | 17 | 2081.72 | 2635.72 |
| | NO: 18) | | | |

*MWT indicates the molecular weight after addition of companion ions.

TABLE 2
Data collected from experiments measuring angiogenic activity
in semi-quantitative/qualitative scale.

| | Samples | | | | | | | | | |
|---|---|---|---|---|---|---|---|---|---|---|
| Treatment | 1 | 2 | 3 | 4 | 5 | 6 | 7 | 8 | Mean | % Diff. |
| Control | 2 | 3 | 2 | 3 | 2 | 3 | | | 2.50 | 0.00 |
| JC15-18N | 3 | 2 | 3 | 3 | | | | | 2.75 | 10.00 |
| (SEQ ID NO: | | | | | | | | | | |
| 28) | | | | | | | | | | |
| JC15-15C | 2 | 3 | 2 | 3 | 2 | 3 | 3 | 4 | 2.75 | 10.00 |
| (SEQ ID NO: 7) | | | | | | | | | | |
| JC15-12N | 4 | 2 | 3 | 2 | 3 | 2 | 3 | | 2.71 | 8.57 |
| (SEQ ID NO: 8) | | | | | | | | | | |
| JC15 | 3 | 2 | 3 | 2 | 3 | | | | 2.60 | 4.00 |
| (SEQ ID NO: 5) | | | | | | | | | | |
| PL-1 | 2 | 3 | 2 | 3 | 2 | 3 | 2 | 3 | 2.50 | 0.00 |
| (SEQ ID NO: | | | | | | | | | | |
| 22) | | | | | | | | | | |
| PF-2 | 2 | 3 | 2 | 3 | | | | | 2.50 | 0.00 |
| SEQ ID NO: 27) | | | | | | | | | | |
| PF-1 | 2 | 3 | 2 | 3 | | | | | 2.50 | 0.00 |
| (SEQ ID NO: | | | | | | | | | | |
| 26) | | | | | | | | | | |
| JC15-10C | 2 | 3 | 2 | 3 | 2 | 3 | 2 | 3 | 2.50 | 0.00 |
| (SEQ ID NO: | | | | | | | | | | |
| 30) | | | | | | | | | | |
| PL-2 | 2 | 3 | 2 | 3 | 2 | 3 | 2 | | 2.43 | −2.86 |
| (SEQ ID NO: 3) | | | | | | | | | | |
| JC15-10N | 1 | 2 | 2 | 3 | 1 | 2 | 2 | | 1.86 | −25.71 |
| (SEQ ID NO: 1) | | | | | | | | | | |
| C-1 | 2 | 2 | 3 | 1 | 2 | 1 | | | 1.83 | −26.67 |
| (SEQ ID NO: | | | | | | | | | | |
| 24) | | | | | | | | | | |

TABLE 3
a presentation of the peptides tested in the Matrigel experiment (Example 3)
showing hydrophobic amino acids with white rectangles and hydrophilic amino
acids as dark rectangles. See also FIGS. 15 and 16.

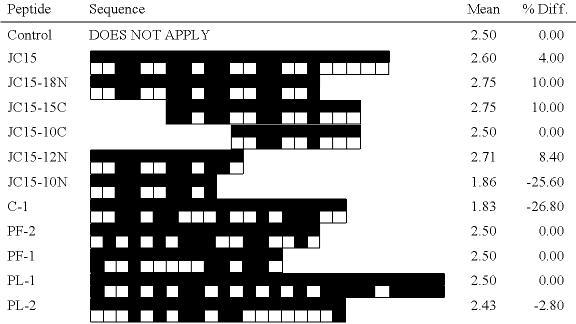

TABLE 4
Amino acid sequences of selected domains derived
from several cytokines, oncostatin and endostatin.

| | | | INTERNAL |
|---|---|---|---|
| SEQ ID NO | DESIGNATION | OLD DESIGNATION | SEQUENCE |
| 37 | CCL5 | RANTES | WVREYINSLE |
| 38 | CCL8 | MCP-2 | WVRDSMKHL |
| 39 | CCL11 | EOTAXIN | KKWVQDSMK |
| 40 | CCL12 | MCP-5 | WVKNSINHL |
| 41 | CCL13 | MCP-4 | WVQNYMKHL |
| 42 | CCL14 | CC-1/CC-3 | KWVQDYIKDM |
| 43 | CCL15 | MIP-5 | LTKKGRQVCA |
| 44 | CCL16 | — | KRVKNAVKY |
| 45 | CCL18 | MIP-4 | LTKRGRQICA |
| 46 | CCL18 | MIP-4 | KKWVQKYIS |
| 47 | CCL19 | MIP-3 BETA | WVERIIQRLQ |
| 48 | CCL23 | MIP-3 | LTKKGRRFC |
| 49 | CCL27 | ESKINE | LSDKLLRKVI |
| 50 | CCL28 | CCK1 | VSHHISRRLL |
| 51 | XCL2 | SCM-1 BETA | WVRDVVRSMD |
| 52 | CX3CL1 | FRACTALKINE | WVKDAMQHLD |
| 53 | CXCL1 | MGSA | MVKKIIEKM |
| 54 | CXCL3 | MIP-2 BETA | MVQKIIEKIL |
| 55 | CXCL4 | PF-4 | LYKKIIKKLL |
| 56 | CXCL5 | ENA-78 | FLKKVIQKIL |
| 57 | CXCL6 | GCP-2 | FLKKVIQKIL |
| 58 | CXCL7 | PRO-PLATELET PRO | IKKIVQKKLA |
| 59 | CXCL8 | IL8 | WVQRVVEKFL |
| 60 | CXCL10 | IP-10 | AIKNLLKAVS |
| 61 | CXCL11 | IP-9 | IIKKVER |
| 62 | CXCL13 | B13 | WIQRMMEVLR |
| 63 | IL10 | — | AVEQVKNAFN |
| 64 | IL5 | — | TVERLFKNLS |
| 65 | IL7 | — | FLKRLLQEI |
| 66 | IL11 | — | LDRLLRRL |
| 67 | IL20 | — | LLRHLLRL |
| 68 | IL22 | — | KDTVKKLGE |
| 69 | IL24 | — | LFRRAFKQLD |
| 70 | IL26 | — | WIKKLLESSQ |
| 71 | ONCO-frag | — | SRKGKRLM |
| 72 | ENDO-frag | F-COLLAGEN XVIII | IVRRADRAAV |

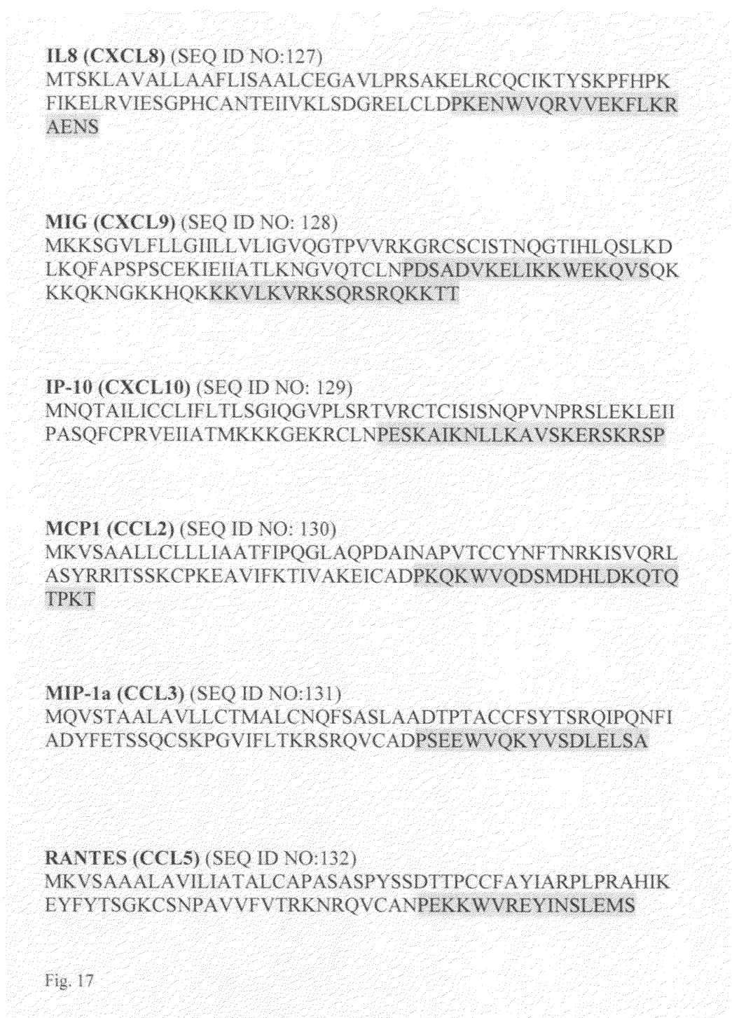

TABLE 5
Chemokines involved in development of various diseases. The sequences of the
chemokines are shown in the sequence listing with the following sequence numbers:
IL8 (SEQ ID NO: 31), MIG (SEQ ID NO: 32), IP-10 (SEQ ID NO: 33), MCP1 (SEQ ID
NO: 34), MIP1a (SEQ ID NO: 35) and Rantes (SEQ ID NO: 36) and in FIG. 17.

| | Chemokine --→ | | | | | |
|---|---|---|---|---|---|---|
| Disease | IL8 | MIG | IP-10 | MCP1 | MIP1a | Rantes |
| Exp Autoimmune Enc | | | 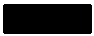 | 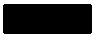 | 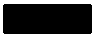 | 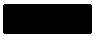 |
| Multiple Sclerosis | | 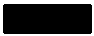 | 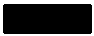 | 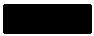 | 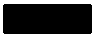 | 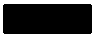 |
| Allografts | 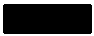 | 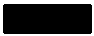 | 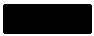 | 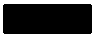 | 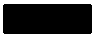 | 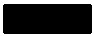 |
| Asthma | | | | 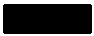 | | 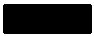 |
| Rheumatoid Arthritis | | | | | | |
| Osteo Arthritis | 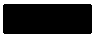 | | 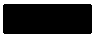 | 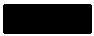 | 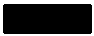 | 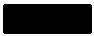 |
| Neoplasia | 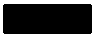 | | 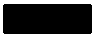 | 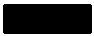 | | 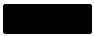 |
| Vascular Disease | 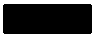 | | 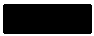 | 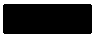 | | 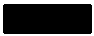 |

TABLE 6
Endothelial Cell migration Experiment

| Treatment | Concentration | | Endothelial Cell Migration |
|---|---|---|---|
| NT | | | 1.00 |
| VEGF | 150 | ng/ml | 0.78 |
| 10N | 100 | nM | 0.42 |
| 10N | 1 | μM | −1.57 |
| 10N | 10 | μM | 0.90 |
| 10N | 100 | μM | 0.80 |
| 10N | 1 | mM | −1.01 |
| NT | | | 1.00 |
| VEGF | 75 | ng/ml | 0.69 |
| 10N | 100 | nM | −1.18 |
| 10N | 1 | μM | 0.57 |
| 10N | 10 | μM | −1.02 |
| 10N | 100 | μM | −1.01 |
| 10N | 1 | mM | 0.55 |
| NT | | | 1.00 |
| VEGF | 150 | ng/ml | 0.77 |
| 10N | 100 | nM | 0.55 |
| 10N | 1 | μM | −1.35 |
| 10N | 10 | μM | −1.02 |
| 10N | 100 | μM | −1.06 |
| 10N | 1 | mM | 0.92 |

1. A lytic peptide having angiogenesis activity wherein said peptide is a molecular fragment derived from a corresponding full-length protein molecule.

2. A lytic peptide fragment according to claim 1, having the capacity to inhibit angiogenesis wherein said peptide fragment has the sequence: FAKKFAKKFK (SEQ ID NO: 1).

3. A lytic peptide fragment according to claim 1, having the capacity to inhibit angiogenesis wherein said peptide fragment has the sequence:
(SEQ ID NO: 2)
IVRRADRAAVPIVNLKDELL.

4. A lytic peptide fragment according to claim 1, having the capacity to inhibit angiogenesis wherein said peptide fragment has the sequence:
(SEQ ID NO: 3)
MFGNGKGYRGKRATTVTGTP.

5. A lytic peptide fragment according to claim 2, having anti-inflammatory properties in a mammal.

6. A method for treating chronic inflammation in a mammal comprising administering to said mammal in need of such treatment an effective amount of the lytic peptide fragment as defined in claim 2 or a pharmaceutically acceptable salt thereof.

7. A method for treatment of chronic inflammation related disorders or conditions selected from among arthritis, ulcerated colitis, Crohn's disease, cancer, multiple sclerosis, cervical spondylosis, tinnitus, systemic lupus, erythematosis, graft rejection, psoriasis, arteriosclerosis, hypertension and is chemia-reperfusion comprising administering to a mammal in need of such treatment a lytic peptide fragment as defined in claim 2 or a pharmaceutically acceptable salt thereof.

8. A pharmaceutical composition for treating disorders or diseases which are ameliorated by the inhibition of angiogenesis comprising treatment of a mammal with an effective amount of a lytic peptide fragment having the sequence: FAKKFAKKFK (SEQ ID NO: 1), IVRRADRAAVPIVNLKDELL (SEQ ID NO: 2), MFGNGKGYRGKRATTVTGTP (SEQ ID NO: 3) or a pharmaceutically acceptable salt thereof.

9. A lytic peptide fragment according to claim 1, having the capacity to accelerate angiogenesis wherein said peptide fragment has the sequence: FAKKFAKKFKKFAKFAFAF (SEQ ID NO: 4), FAKKFAKKFAKKFAK(SEQ ID NO: 6),KKFKKFAKKFAKFAF(SEQ ID NO: 7) or FAKKFAKKFKKF(SEQ ID NO: 8)or a pharmaceutically acceptable salt thereof.

10. A pharmaceutical composition for the treatment of disorders or diseases which are ameliorated by the acceleration of angiogenesis comprising treatment of a mammal with an effective amount of a lytic peptide fragment having the sequence: FAKKFAKKFKKFAKFAFAF (SEQ ID NO: 4), FAKKFAKKFAKKFAK (SEQ ID NO: 6), KKFKKFAKKFAKFAF(SEQ ID NO: 7) or FAKKFAKKFKKF(SEQ ID NO: 8) or a pharmaceutically acceptable salt thereof.

11. A method for treating ulceratve colitis in a mammal comprising administering to said mammal in need of such treatment an effective amount of the lytic peptide as defined in claim 2 or a pharmaceutically acceptable salt thereof.

12. A lytic peptide fragment according to claim 2, having the capacity to modulate inflammatory bowel disease and ulcerative colitis in a mammal.
